(19) United States
(12) Patent Application Publication
(10) Pub. No.: US 2025/0115960 A1
(43) Pub. Date: Apr. 10, 2025

(54) NUCLEIC ACID TESTING METHOD AND SYSTEM

(71) Applicant: BIONOVA (SHANGHAI) MEDICAL TECHNOLOGY CO., LTD., Shanghai (CN)

(72) Inventors: Xiaolan WANG, Pudong Shanghai (CN); Enzhuo YANG, Pudong Shanghai (CN)

(73) Assignee: BIONOVA (SHANGHAI) MEDICAL TECHNOLOGY CO., LTD., Shanghai (CN)

(21) Appl. No.: 18/725,579

(22) PCT Filed: Dec. 30, 2022
(86) PCT No.: PCT/CN2022/143769

§ 371 Date: Jun. 28, 2024

(30) Foreign Application Priority Data
Dec. 31, 2021 (CN) 202111675224.9

(51) Int. Cl. C12Q 1/6883; C12Q 1/6855

(52) U.S. Cl. CPC C12Q 1/6883; C12Q 1/6855; C12Q 2600/154

(57) ABSTRACT

Provided are a nucleic acid testing method and system. Specifically provided is a method for identifying the presence or absence of one or more target nucleic acids in a sample, or for identifying the content thereof.

### CROSS REFERENCE TO RELATED APPLICATIONS

This application is a 35 U.S.C. § 371 National Stage of International Patent Application No. PCT/CN2022/143769, filed Dec. 30, 2022, designating the United States, which claims priority from Chinese Patent Application No. 202111675224.9, filed Dec. 31, 2021, the disclosures of which are incorporated herein in their entirety by reference, and priority is claimed to each of the foregoing.

### REFERENCE TO SEQUENCE LISTING

The instant application contains a Sequence Listing which has been submitted electronically in XML format and is hereby incorporated by reference in its entirety. Said XML copy, created on Dec. 18, 2024, is named “20241213_0145-pa-003 us.xml” and is 45,056 bytes in size.

### TECHNICAL FIELD

The present application relates to a nucleic acid testing method and system. In particular, the present application relates to a test of small amounts of nucleic acids.

### BACKGROUND ART

Specific detection for target nucleic acids is an important tool in diagnostic medicine and molecular biology research. Test for target nucleic acids can be used for, for example, identifying pathogens (e.g., bacteria or viruses) in host subjects, determining normal gene expression and identifying mutant genes (e.g., oncogenes), typing tissues for compatibility before tissue transplantation, analyzing homology between genes of different substances, and/or identifying alleles and polymorphisms of gene variants, and the like.

In these application scenarios, it usually requires the ability for testing and/or identifying a small amount of target nucleic acids in a nucleic acid sample or mixture containing a large amount of non-target nucleic acids.

The test sensitivity or lower limit of test of the currently commonly used test methods are insufficient. For example, a signal from a small amount of target molecules may be masked by signals from a large amount of non-target substances (e.g., background signal or noise), and these “background signals” often need to be removed to improve the lower limit of test. However, in the process of removing the background signal, the signal from the target molecule may be weakened or lost.

In addition, in order to amplify the signal from a small amount of target molecules, it is often necessary to amplify the molecules in the sample. Exponential amplification methods such as PCR often cause amplification bias, thereby affecting the accuracy of the test results. Moreover, when multiple target molecules are involved, performing multiple such exponential amplifications will bring more problems.

Therefore, there is an urgent need to develop more accurate and sensitive nucleic acid test methods, especially for the test of small or ultra-small amounts of target molecules in samples.

### SUMMARY

The present application provides a method and product for identifying target nucleic acids (e.g., a detection kit or a detection system). The method and product according to the present application can significantly improve the lower limit of test, and can sensitively and accurately detect ultra-small amounts of nucleic acids in a sample. For example, the method according to the present application can sensitively and accurately test nucleic acid molecules from circulating tumor cells, such as cfDNA and/or ctDNA. For example, compared to an existing method (e.g., PCR method, such as test using qPCR), the method according to the present application can increase the sensitivity of test by at least 10 times (e.g., at least 20 times, at least 30 times, at least 40 times, at least 50 times, at least 60 times, at least 70 times, at least 80 times, at least 90 times, or even more than at least 100 times). For example, the method according to the present application can accurately test nucleic acid molecules (e.g., DNA) in a sample at a content of 10 ng or less, for example, the nucleic acid molecules in the sample at the content of 9 ng or less, 8 ng or less, 7 ng or less, 6 ng or less, 5 ng or less, 4 ng or less, 3 ng or less, 2 ng or less, 1 ng or less, 0.9 ng or less, 0.8 ng or less, 0.7 ng or less, 0.6 ng or less, 0.5 ng or less, 0.4 ng or less, or less.

The method according to the present application may be an in vitro or ex vivo method. In some cases, the results of identification obtained using the method according to the present application, in combination with other information (e.g., other information of the subject, or other clinical parameters/indicators), are sufficient to derive a clear diagnostic result for the health or disease progression of the subject.

In one aspect, the present application provides a method for identifying the presence or absence of one or more target nucleic acids in a sample, or for identifying the content thereof, including: a) treating the sample under a condition that nucleic acids derived from the sample is capable of being subjected to linear amplification, so as to produce a linear amplification product of the nucleic acids; b) performing exponential amplification on the linear amplification product to produce an exponential amplification product of the nucleic acids; and c) contacting the exponential amplification product of the nucleic acids with an extension primer under extension conditions including terminator nucleotide to produce extended oligonucleotide. The method further includes: d) analyzing the extended oligonucleotide to identify the presence or absence of one or more target nucleic acids in the sample, or to identify the content thereof.

In certain embodiments, step a) includes: contacting the sample with oligonucleotide that is capable of binding to the nucleic acids to form an oligonucleotide hybrid; and contacting the oligonucleotide hybrid with an amplification composition under a condition that linear amplification is allowed to occur, so as to produce the linear amplification product of the nucleic acids.

In certain embodiments, step a) includes: ligating the nucleic acids derived from the sample with a adaptor sequence to form the adaptor-containing nucleic acids; contacting the adaptor-containing nucleic acids with oligonucleotide that is capable of specifically binding to the adaptor sequence to form an oligonucleotide hybrid; and contacting the oligonucleotide hybrid with the amplification composition under a condition that linear amplification is allowed to occur, so as to produce the linear amplification product of the nucleic acids.

In certain embodiments, the adaptor sequence comprises a promoter sequence of RNA polymerase.

In certain embodiments, the adaptor sequence comprises an SP6 promoter sequence, a T7 promoter sequence and/or a T3 promoter sequence.

In certain embodiments, the oligonucleotide that is capable of specifically binding to the adaptor sequence is at least partially complementary to the adaptor sequence.

In certain embodiments, the oligonucleotide that is capable of specifically binding to the adaptor sequence comprises a complementary sequence of the SP6 promoter sequence, a complementary sequence of the T7 promoter sequence, and/or a complementary sequence of the T3 promoter sequence.

In certain embodiments, the adaptor sequence comprises one or more modifications through which the nucleic acid sequence of the adaptor sequence remains unchanged after the adaptor sequence is subjected to bisulfite treatment.

In certain embodiments, the adaptor sequence comprises methylation modification of one or more bases.

In certain embodiments, the cytosine comprised in the adaptor sequence is methylation-modified cytosine.

In certain embodiments, the adaptor sequence is directly or indirectly linked to a 3′ end of the nucleic acids.

In certain embodiments, 5′ end of the adaptor sequence is directly or indirectly linked to a 3′ end of the nucleic acids.

In certain embodiments, ligating the nucleic acids with the adaptor sequence includes: repairing an end of the nucleic acids into a blunt end.

In certain embodiments, ligating the nucleic acids with the adaptor sequence includes: repairing the end of the nucleic acids into the blunt end, and adding deoxyadenosine (dA) to the repaired 3′ end of the sequence.

In certain embodiments, the linear amplification includes a nucleic acid transcription reaction, a strand displacement amplification reaction and/or a rolling circle amplification reaction.

In certain embodiments, the linear amplification is non-target specific amplification not for a specific target.

In certain embodiments, the linear amplification product of the nucleic acids comprises RNA encoded by the nucleic acids.

In certain embodiments, the RNA polymerase is used in the linear amplification.

In certain embodiments, the RNA polymerase includes a T7 RNA polymerase, an SP6 RNA polymerase, and/or a T3 RNA polymerase.

In certain embodiments, step a) further includes: treating the sample by a reagent capable of modifying specific nucleotide which is not methylated to generate other nucleotides.

In certain embodiments, the reagent capable of modifying the specific nucleotide which is not methylated to generate other nucleotides includes a reagent capable of modifying the cytosine which is not methylated to generate uracil.

In certain embodiments, the reagent capable of modifying the specific nucleotide which is not methylated to generate other nucleotides includes bisulfite, β-glucosyltransferase, TET enzyme, pyridine borane and/or A3A deaminase.

In certain embodiments, step a) includes: ligating the nucleic acids derived from the sample with a adaptor sequence to form adaptor-containing nucleic acids, where the adaptor sequence comprises one or more modifications through which the nucleic acid sequence of the adaptor sequence remains unchanged after the adaptor sequence is subjected to bisulfite treatment; treating the adaptor-containing nucleic acids by a reagent containing bisulfite, so as to form converted nucleic acids; contacting the converted nucleic acids with oligonucleotide that is capable of specifically binding to the adaptor sequence to form an oligonucleotide hybrid; and contacting the oligonucleotide hybrid with an amplification composition under a condition that linear amplification is allowed to occur, so as to produce the linear amplification product of the nucleic acids.

In certain embodiments, the method described in the present application is used for identifying the presence or absence of one or more methylated target nucleic acids in a sample, and for identifying the content thereof.

In certain embodiments, the method described in the present application is used for identifying the methylation status of a nucleotide locus in one or more target nucleic acids in a sample.

In certain embodiments, the exponential amplification includes Polymerase Chain Reaction (PCR).

In certain embodiments, b) includes: performing reverse transcription on the linear amplification product, and performing PCR amplification on the reverse transcription product.

In certain embodiments, the exponential amplification includes Reverse Transcriptase Polymerase Chain Reaction (RT-PCR).

In certain embodiments, the exponential amplification is non-target specific amplification not for a specific target.

In certain embodiments, the exponential amplification includes the use of DNA polymerase and/or reverse transcriptase.

In certain embodiments, the terminator nucleotide is modified at the 3′ carbon of the pentose moiety.

In certain embodiments, at least one terminator nucleotide is dideoxynucleotide.

In certain embodiments, the dideoxynucleotide is selected from ddATP, ddGTP, ddCTP, ddTTP and ddUTP.

In certain embodiments, the terminator nucleotide is an acyclic nucleotide.

In certain embodiments, the acyclic nucleotide is selected from acyATP, acyCTP, acyGTP, acyTTP and acy-bromo-UTP.

In certain embodiments, the method can identify 2 or more target nucleic acids in the sample.

In certain embodiments, the terminator nucleotide comprises: 1) terminator nucleotide having specificity to other nucleotides generated by modification of specific nucleotide which is not methylated after the treatment, and 2) terminator nucleotide having specificity to the specific nucleotide which is methylated.

In certain embodiments, the extended oligonucleotide comprises a detectable marker.

In certain embodiments, the detectable marker is a mass marker.

In certain embodiments, the mass marker is a mass distinguishable tag.

In certain embodiments, the terminator nucleotide comprises the mass distinguishable tag.

In certain embodiments, in the method according to the present application, the mass marker is tested by mass spectrometry.

In certain embodiments, the mass spectrometry is Matrix-Assisted Laser Desorption/Ionization (MALDI) mass spectrometry.

In certain embodiments, the nucleic acids derived from the sample comprise cfDNA and/or genomic DNA (gDNA).

In certain embodiments, the nucleic acids derived from the sample comprise ctDNA derived from tumor tissue and/or DNA derived from exfoliated cells of organs or fetuses.

In certain embodiments, the content of the nucleic acids derived from the sample is less than about 10 ng.

In certain embodiments, the content of the nucleic acids derived from the sample is less than about 1 ng.

In certain embodiments, the sample is a biological sample. The biological sample may include blood, body fluids and tissues (for example, organ tissues, formalin-fixed and paraffin-embedded FFPE sample, urines, feces, cerebrospinal fluids, pleural effusions, oral rinses, alveolar lavage fluids, amniotic fluids and the like), and/or an in vitro culture sample.

In certain embodiments, the sample is derived from a subject.

In certain embodiments, the extension primer is capable of specifically binding to the target nucleic acid.

In certain embodiments, the nucleic acid sequence of the extension primer is at least partially complementary to the sequence of the target nucleic acid.

In certain embodiments, the method described in the present application is used for identifying a methylation status of cytosine nucleotide of one or more CpG dinucleotides in nucleic acid molecules.

In certain embodiments, the one or more CpG dinucleotides are positioned in a promoter region of a gene.

In certain embodiments, the method described in the present application is an in vitro or ex vivo method.

In another aspect, the present application provides a method for identifying a biomarker associated with a disease, a disease outcome, and/or a treatment regimen outcome, the method including the following steps: i) identifying the presence or absence of one or more target nucleic acids derived from one or more samples, or identifying the content thereof according to the method described in the present application, the one or more samples being derived from one or more subjects having a known disease, a disease outcome, and/or a treatment regimen outcome; ii) identifying the presence or absence of one or more target nucleic acids derived from one or more samples, or for identifying the content thereof according to the method described in the present application, the one or more samples being derived from a normal subject; and iii) identifying a difference between the presence or absence of one or more target nucleic acids, or the content thereof in step i) and the presence or absence of the one or more target nucleic acids, or content thereof described in step ii), and accordingly identifying the difference as a biomarker associated with the disease, disease outcome, and/or treatment regimen outcome.

In another aspect, the present application provides a method for identifying methylation associated with a disease, a disease outcome, and/or a treatment regimen outcome, the method including the following steps: i) identifying methylated or unmethylated nucleotides in one or more target nucleic acids derived from one or more samples according to the method described in the present application, the one or more samples being derived from one or more subjects having a known disease, a disease outcome, and/or a treatment regimen outcome; ii) identifying methylated or unmethylated nucleotides in one or more target nucleic acids derived from one or more samples according to the method described in the present application, the one or more samples being derived from a normal subject; and iii) identifying a difference between the methylated or unmethylated nucleotides in the one or more target nucleic acids in step i) and the methylated or unmethylated nucleotides in the one or more target nucleic acids in step ii), and accordingly identifying the differentiated methylated or unmethylated nucleotides as methylation associated with the disease, disease outcome, and/or treatment regimen outcome.

In another aspect, the present application provides a kit for identifying the presence or absence of one or more target nucleic acids in a sample, or for identifying the content thereof according to the method described in the present application, the kit comprising: a linear amplification component; an exponential amplification component; and an extension component comprising a terminator nucleotide and an extension primer. In some cases, the extension component comprises the terminator nucleotide, a thermostable elongase, and the extension primer.

In another aspect, the present application provides a kit for identifying the presence or absence of one or more target nucleic acids in a sample, or for identifying the content thereof, the kit comprising: a linear amplification component; an exponential amplification component; and an extension component comprising a terminator nucleotide and an extension primer. In some cases, the extension component comprises the terminator nucleotide, a thermostable elongase, and the extension primer.

In certain embodiments, in the kit described in the present application, the linear amplification component, the exponential amplification component, and the extension component are mixed with each other.

In certain embodiments, in the kit described in the present application, the linear amplification component, the exponential amplification component, and the extension component are each independently present in a separate package.

In certain embodiments, the kit described in the present application comprises one or more of the following components: a) a reagent that may modify specific nucleotide which is not methylated to produce other nucleotides; b) one or more nucleic acid polymerases; c) one or more primers; d) a MALDI matrix compound; and e) a MALDI substrate.

In another aspect, the present application provides a system, comprising the kit described in the present application, and optionally comprising one or more parts selected from the group consisting of a usage instruction, a reagent, and an apparatus for using the kit.

In certain embodiments, in the system described in the present application, the apparatus for using the kit includes a thermal cycler and/or a mass spectrometer.

In the method of the present application, step c) may include: repeating multiple temperature cycles to amplify the amount of extension product in the reaction. For example, the extension reaction can be cycled 2 or more times. For example, the extension reaction can be cycled about 10, 15, 20, 50, 100, 200, 300, 400, 500, or 600 or more times. For example, the extension reaction can be cycled 20 to 50 times. For example, the extension reaction can be cycled at least 50, 55, 60, 65, 70, 75, 80, 85, 90, 95, or 100 times.

Those skilled in the art can easily perceive other aspects and advantages of the present disclosure from the detailed description below. In the detailed description below, only exemplary embodiments of the present disclosure are shown and described. As will be appreciated by those skilled in the art, the content of the present disclosure enables those skilled in the art to make changes to the disclosed specific embodiments without departing from the spirit and scope of the invention to which the present application relates. Accordingly, the descriptions in the drawings and specification of the present application are merely exemplary and not limiting.

### BRIEF DESCRIPTION OF THE DRAWINGS

The specific features of the invention to which the present application relates are set forth in the appended claims. The features and advantages of the invention to which the present application relates can be better understood by reference to the exemplary embodiments described in detail below and the drawings. The accompanying drawings are briefly described as follows:

FIG. 1 shows an experimental process example of a method according to the present application.

FIGS. 2A-2F show nucleic acid mass spectrometric test result (a sample is 1 ng of methylation-modified DNA) of a method of the present application.

FIGS. 3A-3D show an exemplary DNA conversion method.

FIG. 4 shows a linear amplification reaction based on another strand displacement amplification.

FIG. 5 shows a linear amplification reaction based on another rolling circle amplification.

FIG. 6 shows methylation detection accuracy of a test method according to the present application in clinical reaction.

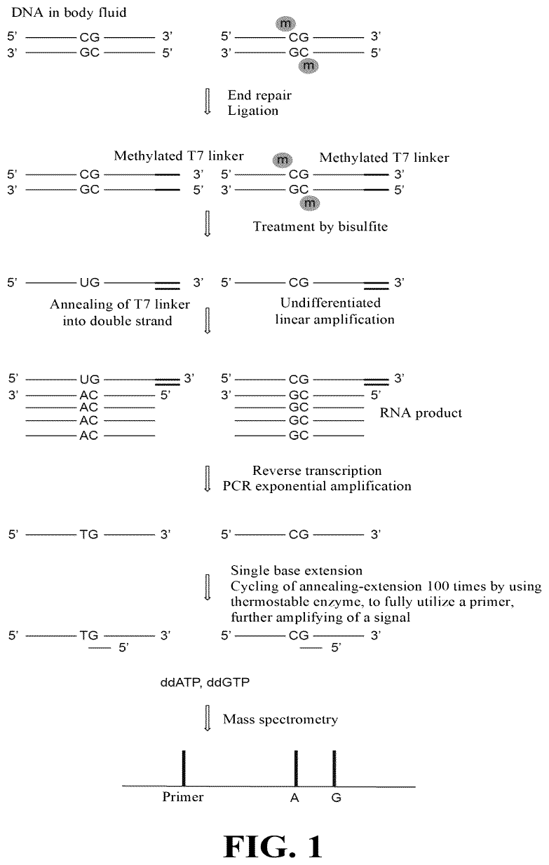

### DETAILED DESCRIPTION

The invention involved in the present application is described below by specific embodiments, and those familiar with this technology can easily understand other advantages and effects of the invention involved in the present application through the disclosed content of this specification.

Unless otherwise stated, the science and technology terms used in the present application have the same meanings commonly understood by the those skilled in the art of molecular biology based on technical documents or other well-known technical publications related to molecular biology, and the technical documents include, for example, Dictionary of Dictionary of Microbiology and Molecular Biology, 2nd ed. (Singleton et al., 1994, John Wiley & Sons, New York, NY) (Singleton et al., “Dictionary of Microbiology and Molecular Biology”, 2nd edition, 1994).

Unless otherwise stated, the technology used or considered in the present application is the standard method well known in the field of molecular biology.

In the present application, the term “about”, when used for specifying certain ranges of values, is not limited to the specified exact values, but may include a value that is different from the specified value. For example, it may include a value that differs by up to 10% from the listed particular values. Thus, the term “about” is used for covering a range of variation of ±10% or less, ±5% or less, ±1% or less, ±0.5% or less, or ±0.1% or less from particular values.

In the present application, the term “sample” is used in its broadest meaning. The term “biological sample” includes, but is not limited to, any number of substances from currently living individuals, or once living individuals. The living individuals include, but are not limited to, humans, mouse, rat, monkeys, dogs, rabbits, and other animals or plants. The substances include, but are not limited to, blood (e.g., whole blood), plasmas, serums, cerebrospinal fluids, tissue fluids (e.g., pleural effusions), lavage fluids (e.g., alveoli), urines, amniotic fluids, synovial fluids, endothelial cells, leukocytes, monocytes, other cells, organs, tissues, bone marrows, lymph nodes, spleens, and the like. The sample may also include living tissues and/or treated tissues, for example biopsy tissues (e.g., liquid biopsy tissues), formalin-fixed and paraffin-embedded (FFPE) tissues and the like.

For example, the sample (e.g., a biological sample) can be a specimen containing a substance of interest to be analyzed, for example a microorganism, virus, nucleic acid (e.g., gene), or components thereof, and the components can contain nucleic acid in an analyte or nucleic acid obtained from the analyte. The sample can be from any source, for example a biological specimen or an environmental source. The biological specimen includes any tissue or material obtained from a living or dead organism, and the tissue or material can contain the analyte, or the nucleic acid in the analyte or the nucleic acid obtained from the analyte. Examples of the biological sample includes respiratory tissues, exudates (e.g., bronchoalveolar lavage fluids), living tissue specimens, sputums, peripheral blood, plasmas, serums, lymph nodes, gastrointestinal tissues, feces, urines, cerebrospinal fluids, tissue fluids (e.g., pleural effusions), lavage fluids (e.g., alveoli), or other fluids, tissues or materials. Examples of the environmental sample include water, ice, soil, suspensions, residues, biofilms, atmospheric dust particles, and aerosols. The samples can be treated specimens or materials, for example obtained by treating the samples using filtration, centrifugation, precipitation, or adsorption to a medium (e.g., a matrix or carrier). Other treatments for the samples can include treatments that physically or mechanically disrupt tissue, cell aggregates or cell, thereby releasing intracellular components including nucleic acids into a solution that can contain other components, such as enzymes, buffers, salts, detergents and the like. The samples can also include tissues, for example biopsy tissues (e.g., liquid biopsy tissues), Formalin-Fixed and Paraffin-Embedded (FFPE) tissues and the like.

In the present application, the term “contact” generally refers to mixing two or more components at least partially with one another. The contact can be achieved by mixing each component in a fluid or semi-fluid mixture. The contact can also be achieved when one or more components are in physical contact with one or more other components on a solid surface, such as a solid tissue slice or matrix.

In the present application, the term “nucleotides” generally includes natural and non-natural nucleotides. The nucleotides include, but are not limited to, naturally occurring nucleoside monophosphate, nucleoside diphosphate and nucleoside triphosphate; deoxyadenosine monophosphate, deoxyadenosine diphosphate and deoxyadenosine triphosphate; deoxyguanosine monophosphate, deoxyguanosine diphosphate and deoxyguanosine triphosphate; deoxythymidine monophosphate, deoxythymidine diphosphate and deoxythymidine triphosphate; deoxycytidine monophosphate, deoxycytidine diphosphate and deoxycytidine triphosphate; deoxyuridine monophosphate, deoxyuridine diphosphate and deoxyuridine triphosphate; and deoxyinosine monophosphate, deoxyinosine diphosphate and deoxyinosine triphosphate (referred to in the present application as dA, dG, dT, dC, dU and dI, or A, G, T, C, U and I in the present application, respectively). The nucleotides also include, but are not limited to, modified nucleotides and nucleotide analogs. The modified nucleotides and nucleotide analogs include, but are not limited to, dideoxynucleotides, acyclic nucleotides, deazapurine nucleotides, for example, 7-deaza-deoxyguanosine (7-deaza-dG) monophosphate, diphosphate and triphosphate and 7-deaza-deoxyadenosine (7-deaza-dA) monophosphate, diphosphate and triphosphate, deuterium-deoxythymidine (deuterium-dT) monophosphate, diphosphate and triphosphate, methylated nucleotides, for example, 5-methyldeoxycytidine triphosphate, 13C/15N labeled nucleotides and deoxyinosine monophosphate, diphosphate and triphosphate. The modified nucleotides, isotope-rich nucleotides, depleted nucleotides, tagged and labeled nucleotides and nucleotide analogs can be obtained using a combination of a variety of functional groups and attachment positions.

In the present application, the term “nucleic acid” generally refers to a polynucleotide compound that includes oligonucleotide, and the oligonucleotide includes nucleoside or a nucleoside analog which has a nitrogen-containing heterocyclic base or base analog covalently linked by a standard phosphodiester bond or other bonds. The nucleic acid includes RNA, DNA, a chimeric DNA-RNA polymer, or analogs thereof. In the nucleic acid, the backbone may be composed of a plurality of bonds, including one or more of a sugar phosphodiester bond, a Peptide Nucleic Acid (PNA) bond, a phosphorothioate bond, a methylphosphate bond, or combinations thereof. The glycosyl moieties in the nucleic acid may be ribose, deoxyribose, or a similar compound having substituents, for example, 2′ methoxy and 2′ halide (e.g., 2′-F) substituents. The nitrogen-containing base may be a conventional base (A, G, C, T, or U), an analog thereof (e.g., inosine), a derivative of a purine or pyrimidine base (e.g., N4-methyldeoxyguanosine, deaza- or aza-purine, deaza- or aza-pyrimidine, pyrimidine or purine having altered or replaced substituents at any of a plurality of chemical positions (e.g., 2-amino-6-methylaminopurine, 06-methylguanine, 4-thio-pyrimidine, 4-amino-pyrimidine, 4-dimethylhydrazine-pyrimidine, and 04-alkyl-pyrimidine), or a pyrazole compound, for example unsubstituted or 3-substituted pyrazol [3,4-d]pyrimidine). The nucleic acid may include a “abasic” position, where the backbone does not have a nitrogen-containing base at one or more positions, e.g., one or more abasic positions may form a adaptor region that joins individual oligonucleotide sequences together. The nucleic acid may comprise only conventional glycosyl, bases, and bonds as present in conventional RNA and DNA, or may comprise conventional components and substituents (e.g., conventional bases linked by a 2′ methoxy backbone, or a polymer containing a mixture of conventional bases and one or more base analogs). The term includes “Locked Nucleic Acid” (LNA) that may comprise one or more LNA nucleotide monomers, having a bicyclic furanose unit locked in an RNA-mimicking sugar conformation that enhances the hybridization affinity to complementary sequences in ssRNA, ssDNA, or dsDNA.

In addition, the nucleic acid referred to as “polynucleotide” generally refers to two or more nucleotide or nucleotide analogs linked by covalent bonds. The nucleic acid may be any type of nucleic acid suitable for use in the method described in the present application. In certain embodiments, the nucleic acid may be DNA (for example, complementary DNA (cDNA), genomic DNA (gDNA), plasmid and vector DNA, and the like), RNA (for example, viral RNA, messenger RNA (mRNA), short inhibitory RNA (siRNA), ribosomal RNA (rRNA), tRNA and the like), and/or DNA or RNA analogs (for example, including base analogs, saccharide analogs and/or non-natural skeletons, and the like). The nucleic acid may be any form (for example, linear, cyclic, supercoiled, single stranded, double stranded and the like) that may be used for performing the method described in the present application. In certain embodiments, the nucleic acid may be or may be derived from plasmids, phages, Autonomous Replication Sequences (ARS), centromeres, artificial chromosomes, chromosomes, cells, nuclei, or cytoplasms of the cells. In certain embodiments, the nucleic acid is from a single chromosome (for example, a nucleic acid sample may be from one chromosome of a sample obtained from a diploid organism). In the case of fetal nucleic acids, the nucleic acids may be from a paternal allele, a maternal allele, or both a maternal and paternal allele.

In the present application, the terms “oligonucleotide” and “oligomer” are interchangeably used, generally referring to nucleic acid polymers generally composed of less than 1,000 nucleotides (nt), including polymers having a length of about 2 nt to about 900 nt. For example, the oligonucleotide may comprise 5 nt to 500 nt, for example, may comprise 10 nt to 150 nt. For example, the oligonucleotide may be prepared by synthesis using any well known in vitro chemical or enzymatic method, and may be purified after synthesis using standard methods, including for example High Performance Liquid Chromatography (HPLC). In the present application, representative oligonucleotide includes, for example, primers, promoters, detection probe oligonucleotides, target capture oligonucleotides and the like.

In the present application, the term “primers” generally refers to oligonucleotide, of which at least the 3′ end is complementary to a nucleic acid template and which complexes with the template (for example, by hydrogen bonding or hybridization) to obtain a primer-template complex, which is suitable for initiating synthesis by an RNA- or DNA-dependent nucleic acid polymerase (e.g., DNA polymerase or RNA polymerase). The primer may be extended by adding a covalently bonded nucleotide base to the 3′ end of the primer, which base is complementary to the template. The primer extension product is obtained. In the present application, the length of the primers may be at least 10 nucleotides, and may be extended to a length of about 15, 20, 25, 30, 35, 40, 45, 50 or more nucleotides.

In the present application, the term “tagged oligonucleotide” generally refers to oligonucleotide comprising at least a first region and a second region, where the first region comprises a “target hybridization sequence”, the target hybridization sequence being capable of hybridizing to the target nucleic acid sequence of interest; and where the second region comprises a “tag sequence” located at the 5′ end of the target hybridization sequence and incapable of stably hybridizing or binding to the target nucleic acid comprising the target nucleic acid sequence. The hybridization of the target hybridization sequence to the target nucleic acid sequence results in a “tagged target nucleic acid sequence”. The “tag sequence” or “heterologous tag sequence” may be substantially any sequence, with the proviso that the sequence is incapable of stably hybridizing to the target nucleic acid sequence of interest. For example, the tag sequence is incapable of stably hybridizing to any sequence obtained from the genome of an organism to be tested, or to any target nucleic acid under reaction conditions. The tag sequence present in the tagged oligonucleotide is designed to be not to substantially impair or hinder the ability of the target hybridization sequence to hybridize to its target sequence. Furthermore, the tag sequence may have a sufficient length so that once the complementary sequence of the tag sequence is incorporated into the initial DNA primer extension product, a tag-specific primer may then be used for participating in subsequent amplification. The tag sequences of the present application are generally at least 10 nucleotides in length and may be extended to a length of, for example, 15, 20, 25, 30, 35, 40, 45, 50 or more nucleotides. In some cases, the tagged oligonucleotide is a “tagged primer” comprising a tag sequence and a target hybridization sequence. In other cases, the tagged oligonucleotide is a “tagged promoter oligonucleotide” comprising the tag sequence, target hybridization sequence, and promoter sequence; and the promoter sequence may be located at the 5′ end of the tag sequence and act to initiate transcription from the 5′ end.

In the present application, the term “amplification” of the target nucleic acid generally refers to a process of constructing in vitro a nucleic acid chain that is identical or complementary to at least a part of a target nucleic acid sequence or a universal or tag sequence that is an alternative sequence for the target nucleic acid sequence, and this process can occur only when the target nucleic acid is present in a sample. Generally, nucleic acid amplification is to use one or more nucleic acid polymerases and/or transcriptases to produce multiple copies of target polynucleotide or fragment thereof, or multiple copies of a sequence complementary to the target polynucleotide or fragment thereof, or multiple copies of a universal sequence or tag sequence that has been introduced into a amplification system to serve as an alternative sequence for the target polynucleotide, wherein the universal sequence or tag sequence is used, for example, in a test step, to indicate the presence of the target polynucleotide at some point in the test, or for serving as a site for further initiation in the amplification reaction, or for sequencing-related processes or sequencing reactions. The in vitro nucleic acid amplification technology includes transcription-related amplification methods, for example Transcription-Mediated Amplification (TMA) or Nucleic Acid Sequence-Based Amplification (NASBA), and other methods, for example Polymerase Chain Reaction (PCR), Reverse Transcription-PCR (RT-PCR), replicase-mediated amplification, and Ligase Chain Reaction (LCR).

In the present application, the term “linear amplification” refers to an amplification process designed to increase the production of target nucleic acids in a linear proportion to the content of the target nucleic acid in a reaction. For example, a transcription-related reaction may be used for preparing multiple RNA copies from a target DNA, where the increase in the number of copies may be described by a linear factor (e.g., the starting copies of a template×100). For example, in a multi-stage amplification procedure, the linear amplification of the first stage may cause the starting number of target nucleic acid strands or complementary strands of the target nucleic acid strands to increase by at least 10 times, e.g., at least 100 times, e.g., 10 to 1000 times, before the start of the amplification reaction of the second stage. One example of the linear amplification system is T7-based DNA linear amplification. Other methods are described in the present application. Thus, the term “linear amplification” refers to an amplification reaction that does not result in exponential amplification of the target nucleic acid sequence. The term “linear amplification” does not refer to a method of preparing only a single copy of the nucleic acid strand. For example, the linear amplification may be based on known reactions including nucleic acid transcription reactions, strand displacement amplification reactions, and/or rolling circle amplification reactions and the like.

In the present application, the term “exponential amplification” generally refers to an amplification process designed to increase the production of target nucleic acids in a geometric proportion to the amount of the target nucleic acid in a reaction. For example, PCR produces a DNA strand for each original target strand and for each synthetic strand present. Similarly, transcription-related amplification produces multiple RNA transcripts for each original target strand and for each subsequently synthesized strand. The amplification is exponential in that the synthesized strands are used as templates in subsequent rounds of amplification. The amplification reaction need not actually produce an exponential increasing amount of nucleic acids to be considered as exponential amplification, as long as the amplification reaction is designed to produce such an increase.

In the present application, the “transcription-related amplification” method is to amplify a target sequence by generating multiple transcripts from a nucleic acid template. Such methods generally use one or more oligonucleotides, one of which provides promoter sequences, deoxyribonucleoside triphosphates (dNTPs), ribonucleoside triphosphates (rNTPs), and an enzyme having RNA and DNA polymerase activity to generate a functional promoter sequence close to the target sequence, and then transcribe the target sequence from the promoter. Examples of transcription-related amplification include Transcription-Mediated Amplification (TMA), Nucleic Acid Sequence-Based Amplification (NASBA), single primer transcription-related amplification, and self-sustained sequence amplification (3SR). Those of ordinary skill in the art will appreciate that alternative amplification methods based on polymerase-mediated extension of oligonucleotide sequences may also be used with the compositions and/or method steps described in the present application.

In the present application, single primers (rather than pairs of primers) can be used to amplify nucleic acids in vitro by preparing transcripts that indicate the presence of the target nucleic acid. For example, the single primer method can use an oligonucleotide primer and optionally a blocker molecule (e.g., a terminator oligonucleotide) to terminate extension of DNA from the target strand. The method can synthesize the multiple copies of the target sequence by treating the target nucleic acid comprising the target sequence with the following substances: 1) an oligonucleotide primer that hybridizes to the 3′ end of the target sequence such that a primer extension reaction can be initiated from the 3′ end, and 2) a blocker molecule that binds to a target nucleic acid adjacent to the 5′ end of the target sequence or near the 5′ end of the target sequence. The oligonucleotide primer is extended in the primer extension reaction using the DNA polymerase to obtain a DNA primer extension product that is complementary to the target sequence, wherein the DNA primer extension product has a 3′ end determined by the blocker molecule and complementary to the 5′ end of the target sequence.

In another example, multiple copies of the target sequence are synthesized by hybridizing a primer to a target DNA at a position in the 3′ portion of the target sequence and hybridizing a primer to a terminator nucleotide at a position in the 5′ portion of the target sequence.

In some cases, examples of single primer transcription-related amplification methods do not require the use of the terminator (oligo) nucleotide.

In the present application, the term “target nucleic acid” generally refers to any nucleic acid that comprises the sequence to be tested. The target nucleic acid may be DNA or RNA. The target nucleic acid may be of any source, for example genomic DNA, mRNA, cDNA, cfDNA, ctDNA and the like. The target nucleic acid may be naturally occurring or synthetic (e.g., amplification products, carriers and the like). The target nucleic acid may be, but need not be, purified or isolated. Depending on the nature of the desired assay, the target nucleic acid may be derived from a plant or animal tissue, or taken from a reaction mixture. The target nucleic acid is not limited in length, although the target nucleic acid may be exposed to a restriction endonuclease prior to test or identification by the method of the present application.

In the present application, the term “marker” generally refers to a molecular moiety or compound that can be tested or produce a detectable response, which the molecular moiety or compound may be directly or indirectly conjugated to the labeled nucleic acid molecules. Direct labeling can use a bond or interaction to link the marker and the nucleic acid molecule to be labeled, the bond or interaction including covalent bonds, non-covalent interactions (hydrogen bonds, and hydrophobic and ionic interactions), or chelates or coordination complexes. Indirect labeling can use directly or indirectly labeled bridging moieties or linkers (e.g., antibodies, oligonucleotides or other compounds) that can amplify the signal. The markers include any detectable moiety. Examples of useful detectable moieties include radionuclides, ligands (e.g., biotin or avidin), enzymes, enzyme substrates, reactive groups, chromophores (detectable dyes, particles or beads), fluorophores, or luminescent compounds (e.g., bioluminescent markers, phosphorescent markers, or chemiluminescent markers).

In the present application, the term “isolation” or “purification” generally refers to removing one or more components from a mixture (e.g., a sample) to isolate from one or more other components in the mixture. Sample components include nucleic acids, and can include cell fragments, proteins, carbohydrates, lipids, and other compounds.

In the present application, the term “multiple target nucleic acids” generally refers to more than one target nucleic acid. In certain embodiments, the plurality of target nucleic acids can be about 2 to about 10000 nucleic acids, about 2 to about 1000 nucleic acids, about 2 to about 500 nucleic acids, or, for example, about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 80, 85, 90, 95, 100, 125, 150, 175, 200, 225, 250, 275, 300, 325, 350, 375, 400, 425, 450, 475, 500, 600, 700, 800, 900, 1000, 2000, 3000, 4000, 5000, 6000, 7000, 8000, 9000 or 10000 target nucleic acids. In certain embodiments, the multiple target nucleic acids are in one or more reaction vessels, and each reaction vessel comprises more than one target nucleic acid. In certain embodiments, the multiple target nucleic acids are in one reaction vessel. In certain embodiments, the multiple target nucleic acids are about 2 to about 100 target nucleic acids. In certain embodiments, the about 2 to about 100 target nucleic acids are in a single reaction vessel.

In the present application, the term “hybridization sequence” generally refers to nucleotide sequences in oligonucleotides that are capable of specifically hybridizing to amplification products or portions thereof, target nucleic acids or portions thereof, target nucleic acids or portions thereof, target nucleic acid variants or portions thereof or complements thereof. The hybridization sequences are easy to design and select, and the sequences can be of a length suitable for hybridization with the amplification products, target sequences or complementary molecules thereof described in the present application. In certain embodiments, the hybridization sequences in each oligonucleotide are about 5 to about 200 nucleotides in length (e.g., about 5-10, about 10-15, about 15-20, about 20-25, about 25-30, about 30-35, about 35-40, about 40-45 or about 45-50, about 50-70, about 80-90, about 90-110, about 100-120, about 110-130, about 120-140, about 130-150, about 140-160, about 150-170, about 160-180, about 170-190, and about 180-200 nucleotides in length).

In the present application, the term “hybridization condition” generally refers to a condition under which two nucleic acids having complementary nucleotide sequences are capable of interacting with each other. The hybridization condition can be highly stringent, moderately stringent or lowly stringent, and the condition for the degree of stringency of these changes is known. Depending on the application of interest, hybridization conditions that allow amplification and/or extension are generally selected.

In the present application, the “specific hybridization” refers to hybridization with some amplification product or target nucleic acid in a sample without substantially hybridizing with other amplification product substances or target nucleic acids in the sample.

In the present application, the term “terminator nucleotide” may be used interchangeably with “termination nucleotide”, “chain terminator reagent” or “chain terminator”, and generally refers to molecules that stop an extension reaction when added to the extension reaction. The chain terminator may include a nucleotide analog that prevents further extension of a polynucleotide chain or oligonucleotide when present in the polynucleotide chain or oligonucleotide. In certain embodiments, the terminator nucleotide is terminator nucleotide. In certain embodiments, the terminator nucleotide is modified nucleotide that, when incorporated into the 3′ end of the nucleic acid molecules (e.g., oligonucleotide) in an extension reaction, will not allow the nucleotide to be further incorporated into the oligonucleotide. In certain embodiments, the terminator nucleotide is not removed from the oligonucleotide or polynucleotide chain in the presence of an enzyme having 3′-5′ exonuclease activity. In certain embodiments, the '3OH of the nucleotide pentose may be substituted with a moiety that produces chain-terminating nucleotide and is also resistant to removal by the enzyme having the 3′-5′ exonuclease activity. In certain embodiments, the pentose 3′ position of the terminator nucleotide is modified to replace OH with another moiety, including, but not limited to, a phosphoryl group, an acetyl group, a 3′-O-methyl group, a 3′-O-(2-nitrobenzyl), a 3′-O-allyl group, a 3′-azido group, and a 3′-amino group. In certain embodiments, the 3′OH group is substituted with hydrogen. In certain embodiments, the modified nucleotide is dideoxynucleotide. In certain embodiments, the modified nucleotide is an acyclic nucleotide. Examples of the terminator nucleotide include a dideoxynucleotide, for example ddA (dideoxyadenine), ddT (dideoxythymine), ddC (dideoxycytosine), ddG (dideoxyguanine), and ddU (dideoxyuracil) and an acyclic nucleotide, for example, acyATP, acyCTP, acyGTP, acyTTP, and acy-bromo-UTP.

In the present application, the term “signal-to-noise ratio” generally refers to quantitatively measuring signal quality by quantifying the ratio of signal to noise intensity when using a test process (e.g., mass spectrometry). In certain embodiments, the intensity peak on one spectrum has a higher signal-to-noise ratio than the low intensity peak produced by the same analyte (e.g., extended oligonucleotide species) on another spectrum. For example, when using mass spectrometry. In the present application, the term “signal” used in the “signal-to-noise ratio” refers to the intensity of the signal peak of the extended oligonucleotide species. In certain embodiments, the method of the present application includes contacting the oligonucleotide hybrid with the extension composition under extension conditions, the extension composition comprising one or more terminator nucleotides.

In the present application, the term “sensitivity” generally refers to the amount of analyte that can detect a given signal-to-noise ratio when using a test process (e.g., mass spectrometry). In certain embodiments, sensitivity can be improved by reducing background noise levels.

In the present application, the term “cell-free DNA (cfDNA)” generally refers to DNA in a sample, which when collected, is not contained within a cell. The term does not refer to DNA that is cell-free by in vitro disruption of a cell or tissue. The cfDNA can include both DNA derived from normal cells and cancer cells. The cfDNA is generally obtained from blood or plasma (“circulation”). The cfDNA can be released into circulation by secretion or cell death processes, such as cell necrosis or apoptosis. For example, some cfDNA can be ctDNA.

In the present application, the term “circulating tumor DNA (ctDNA)” or “circulating cancer DNA” refers to a cell-free DNA (cfDNA) component derived from a tumor.

In one aspect, the present application provides a method for identifying the presence or absence of one or more target nucleic acids in a sample, or for identifying the content thereof. The method according to the present application may include: a) treating the sample under a condition that nucleic acids derived from the sample can be subjected to linear amplification, so as to produce a linear amplification product of the nucleic acids. The method may further include: b) performing exponential amplification on the linear amplification product to produce an exponential amplification product of the nucleic acids. The method may further include: c) contacting the exponential amplification product of the nucleic acids with an extension primer under extension conditions including terminator nucleotide to produce extended oligonucleotide. The method may further include: d) analyzing the extended oligonucleotide to identify the presence or absence of one or more target nucleic acids in the sample, or to identify the content thereof.

In another aspect, the present application provides a method for identifying a biomarker associated with a disease, a disease outcome, and/or a treatment regimen outcome. The method may include the following steps: i) identifying the presence or absence of one or more target nucleic acids derived from one or more samples, or identifying the content thereof according to the method described in the present application, the one or more samples being derived from one or more subjects having a known disease, a disease outcome, and/or a treatment regimen outcome; ii) identifying the presence or absence of one or more target nucleic acids derived from one or more samples, or for identifying the content thereof according to the method described in the present application, the one or more samples being derived from a normal subject; and iii) identifying a difference between the presence or absence of one or more target nucleic acids, or the content thereof in step i) and the presence or absence of the one or more target nucleic acids, or content thereof in step ii), and accordingly identifying the difference as a biomarker associated with the disease, disease outcome, and/or treatment regimen outcome.

In another aspect, the present application provides a method for identifying methylation associated with a disease, a disease outcome, and/or a treatment regimen outcome. The method includes the following steps: i) identifying methylated or unmethylated nucleotides in one or more target nucleic acids derived from one or more samples according to the method described in the present application, the one or more samples being derived from one or more subjects having a known disease, a disease outcome, and/or a treatment regimen outcome; ii) identifying methylated or unmethylated nucleotides in one or more target nucleic acids derived from one or more samples according to the method described in the present application, the one or more samples being derived from a normal subject; and iii) identifying a difference between the methylated or unmethylated nucleotides in the one or more target nucleic acids described in step i) and the methylated or unmethylated nucleotides in the one or more target nucleic acids described in step ii), and accordingly identifying the differentiated methylated or unmethylated nucleotides as methylation associated with the disease, disease outcome, and/or treatment regimen outcome.

In another aspect, the present application provides a kit for identifying the presence or absence of one or more target nucleic acids in a sample, or for identifying the content thereof according to the method described in the present application. The kit may comprise: a linear amplification component; an exponential amplification component; and an extension component comprising a terminator nucleotide and an extension primer.

In another aspect, the present application provides a kit for identifying the presence or absence of one or more target nucleic acids in a sample, or for identifying the content thereof. The kit may comprise: a linear amplification component; an exponential amplification component; and an extension component comprising a terminator nucleotide and an extension primer. For example, the extension component may comprise the terminator nucleotide, a thermostable elongase, and the extension primer.

In another aspect, the present application provides a system. The system may comprise the kit described in the present application, and optionally comprise one or more parts selected from the group consisting of a usage instruction, a reagent, and an apparatus for using the kit.

### Samples and Target Nucleic Acids

The method and product (e.g., the kit, system and the like) of the present application may be used for identifying the presence or absence of one or more target nucleic acids in a sample, or for identifying the content thereof.

In the present application, the identified nucleic acid can be oligonucleotide or polynucleotide, including but not limited to natural nucleic acid (for example, deoxyribonucleic acid (DNA), ribonucleic acid (RNA)), synthetic nucleic acid, and non-natural nucleic acid (for example, peptide nucleic acid (PNA)), unmodified nucleic acid, modified nucleic acid (for example, methylated DNA, labeled nucleic acid, and nucleic acid molecules with one or more modified nucleotides) and the like. In the present application, the “polynucleotide” refers to two or more nucleotides or nucleotide analogues linked through covalent bonds. The nucleic acid can be any type of nucleic acid suitable for the method described in the present application. For example, the nucleic acid can be DNA (for example, complementary DNA (cDNA), genomic DNA (gDNA), cfDNA, ctDNA, plasmid, vector DNA and the like), and can also be nucleic acid analogues (for example, including base analogues, saccharide analogues and/or non-natural skeletons, and the like). The nucleic acid may be any form (for example, linear, cyclic, supercoiled, single stranded, double stranded and the like) that may be used for performing the method described in the present application. For example, the nucleic acid can be or can be from plasmid, bacteriophage, Autonomous Replication Sequence (ARS), centromere, artificial chromosome, chromosome, cell, nucleus or cytoplasm of the cell. In some cases, the nucleic acid can be from a single chromosome (for example, a nucleic acid sample can be from one chromosome of a sample obtained from diploid organisms). In the case of fetal nucleic acids, the nucleic acids may be from a paternal allele, a maternal allele, or both a maternal and paternal allele. For example, the nucleic acid described in the present application can be cell-free DNA, for example cfDNA in a tissue or sample, such as ctDNA from a tumor or cancerous tissue/site.

In the present application, the target nucleic acid molecule to be tested or identified may be of any length. For example, it may comprise at least 1 (e.g., it may comprise at least 2, 3, 4, 5, 10, 15, 20, 25, 30, 35, 40, 45, 50, 55, 60, 65, 70, 75, 80, 85, 90, 95, 100 or more than 100) nucleotides.

In the present application, the target nucleic acid molecule to be tested or identified may comprise one or more modifications, for example, it may comprise one or more deletions, additions, or substitutions of one or more methyl groups or methylation statuses, one or more phosphate groups, one or more acetyl groups, and one or more nucleotides. Examples of one or more deletions, additions, or substitutions of one or more nucleotides include, but are not limited to, the presence or absence of a particular mutation, the presence or absence of a nucleotide substitution (e.g., single nucleotide polymorphism (SNP)), the presence or absence of a repetitive sequence (e.g., dinucleotide, trinucleotide, tetranucleotide, pentanucleotide repeat), the presence or absence of a marker (e.g., a microsatellite), and the presence or absence of a distinguishing sequence (e.g., a sequence that distinguishes one organism from another organism (e.g., a sequence that distinguishes one virus strain from another virus strain). Different nucleic acids of the target nucleic acid and different target nucleic acids may be distinguished in any known manner, for example by quality, binding, distinguishable tags and the like, as described in the present application.

In certain embodiments, variants of the target nucleic acid may be present in the sample at substantially equal (e.g., SNP) frequencies or copy numbers. In certain embodiments, variants of the target nucleic acid may be present in the sample at different frequencies or copy numbers. In certain embodiments, one variant may be present in greater abundance than the other variants. In certain embodiments, the variant with greater abundance is referred to as a wild type, and the variant with smaller abundance is referred to as a mutant. In some cases, the target nucleic acid comprises first and second variants, where the first or second variant shows greater abundance than other variants, i.e., a high-abundance variant and a low-abundance variant, or a major variant and a minor variant. When compared to another variant, the variant showing greater abundance is typically present at a higher concentration or is represented by a higher number of molecules (e.g., copies). The higher concentration may be 2 times or more. In certain embodiments, the higher concentration is 10 times or more. In certain embodiments, the higher concentration is 100 times, 1,000 times, or 10,000 times or more. In certain embodiments, one variant represents a wild type sequence, and the concentration is 100 times or more higher than the other variant. In certain embodiments, the concentration of the variant (low-abundance variant) is significantly lower than that of the other variant (e.g., wild type, high-abundance variant).

In certain embodiments, the method of the present application can be used for testing the presence or absence of a low-abundance target nucleic acid (e.g., cfDNA or ctDNA) in an amount of less than 30%, 20%, 15%, 10%, 8%, 5%, 4%, 3%, 2%, 1%, 0.8%, 0.75%, 0.5%, 0.1%, 0.05%, 0.01% or less of the total amount of nucleic acid in the sample. In certain embodiments, the method of the present application can be used for testing the presence or absence of the low-abundance target nucleic acid (e.g., cfDNA or ctDNA) in an amount of about 1% to about 10% of the total amount of nucleic acid in the sample. In certain embodiments, the method of the present application can be used for testing the presence or absence of the low-abundance target nucleic acid (e.g., cfDNA or ctDNA) in an amount of about 5% or less of the total amount of nucleic acid in the sample. In certain embodiments, the method of the present application can be used for testing the presence or absence of the low-abundance target nucleic acid (e.g., cfDNA or ctDNA) in an amount of about 5% to about 0.75% of the total amount of nucleic acid in the sample. In certain embodiments, the method of the present application can be used for testing the presence or absence of the low-abundance target nucleic acid (e.g., cfDNA or ctDNA) in an amount of about 5% to about 0.1% of the total amount of nucleic acid in the sample. In certain embodiments, the method of the present application can be used for testing the presence or absence of the low-abundance target nucleic acid (e.g., cfDNA or ctDNA) in an amount of about 1% or less of the total amount of nucleic acid in the sample. In certain embodiments, the method of the present application can be used for testing the presence or absence of the low-abundance target nucleic acid (e.g., cfDNA or ctDNA) in an amount of about 0.1% to about 0.001% of the total amount of nucleic acid in the sample.

For example, the content of the target nucleic acids (e.g., DNA, like cfDNA, ctDNA and the like) in a sample can be less than about 100 ng (e.g., less than about 90 ng, less than about 80 ng, less than about 70 ng, less than about 60 ng, less than about 50 ng, less than about 40 ng, less than about 30 ng, less than about 20 ng, less than about 15 ng, less than about 10 ng, less than about 9 ng, less than about 8 ng, less than about 7 ng, less than about 6 ng, less than about 5 ng, less than about 4 ng, less than about 3 ng, less than about 2 ng, less than about 1 ng, less than about 0.9 ng, less than about 0.8 ng, less than about 0.7 ng, less than about 0.6 ng, less than about 0.5 ng, less than about 0.45 ng, less than about 0.4 ng, less than about 0.35 ng, less than about 0.3 ng, less than about 0.2 ng or less).

For example, the content of the target nucleic acids (e.g., DNA, such as cfDNA, ctDNA, etc.) in a sample can be less than about 20% of the total nucleic acid content, e.g., about 15% or less, about 10% or less, about 9% or less, about 8% or less, about 7% or less, about 6% or less, about 5% or less, about 4% or less, about 3% or less, about 2% or less, about 1% or less, about 0.9% or less, about 0.8% or less, about 0.7% or less, about 0.6% or less, about 0.5% or less, about 0.4% or less, about 0.3% or less, about 0.2% or less, about 0.1% or less, about 0.05% or less, about 0.04% or less, about 0.03% or less, about 0.02% or less, about 0.01% or less, or less. The percentages can be mass percentages, volume percentages, and/or molar percentages.

For example, the sample can comprise a mixture of one or more target nucleic acids (each target nucleic acid can have low-abundance and high-abundance variants), or a mixture can be formed by combining one or more samples comprising one or more target nucleic acids (each target nucleic acid can have low-abundance and high-abundance variants). The low-abundance variant can be a variant of the high-abundance variant and can include but is not limited to a mutant (low-abundance variant) of a wild type (high-abundance variant) allele, a variant of a gene present in more than one host (for example, a virus oncogene (low-abundance gene) which is a variant of a normal healthy gene (high-abundance variant)), polymorphisms, including Single Nucleotide Polymorphisms (SNPs), insertions, deletions or other mutant forms of high-abundance variants.

The method of the present application can be used for simultaneously identifying multiple target nucleic acids that may be present in the sample. The multiple target nucleic acids may refer to more than one target nucleic acid. For example, the multiple target nucleic acids may be about 2 to about 10000 target nucleic acids, about 2 to about 1000 nucleic acids, about 2 to about 500 nucleic acids, or about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 80, 85, 90, 95, 100, 125, 150, 175, 200, 225, 250, 275, 300, 325, 350, 375, 400, 425, 450, 475, 500, 600, 700, 800, 900, 1000, 2000, 3000, 4000, 5000, 6000, 7000, 8000, 9000, or 10000 target nucleic acids. In certain embodiments, the multiple target nucleic acids are located in one or more reaction vessels, and each reaction vessel may comprise more than one target nucleic acid. In certain embodiments, the multiple target nucleic acids are located in the same reaction vessel. In certain embodiments, the multiple target nucleic acids are about 2 to about 200 target nucleic acids. In certain embodiments, the about 2 to about 200 target nucleic acids are located in a single reaction vessel.

For example, the nucleic acid test or identification provided by the method described in the present application may be used for testing the presence or absence of methylation or the methylation status at one or more sites (e.g., about 2 to about 10000 sites, about 2 to about 1000 sites, about 2 to about 500 sites, or about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 80, 85, 90, 95, 100, 125, 150, 175, 200, 225, 250, 275, 300, 325, 350, 375, 400, 425, 450, 475, 500, 600, 700, 800, 900, 1000, 2000, 3000, 4000, 5000, 6000, 7000, 8000, 9000, or 10000 sites, which may include different sites of the same nucleic acid molecules, or may include same sites in different nucleic acid molecules) in DNA (particularly cfDNA, like ctDNA).

In the present application, the samples comprising the nucleic acids (e.g., target nucleic acids) may be derived from one or more sources and may comprise a mixture of target nucleic acids, each of which may have one or more high-abundance variants and low-abundance variants of different copy numbers. The samples may also be combined to produce a mixture comprising different target nucleic acids, and the different target nucleic acids may have different abundances, and/or different copy numbers.

For example, the samples of the present application may be collected from an organism, a mineral or geological site (e.g., soil, rock, mineral deposit, and fossil) or a forensic site (e.g., crime scene, contraband or suspected contraband). Thus, the source may be an environmental, for example geological, agricultural, battlefield or soil sources. The source may also be any type of organism, such as any plant, fungus, protozoa, prokaryote, virus or animal, including but not limited to: humans, non-humans, mammals, reptiles, cattles, cats, dogs, goats, pigs, monkeys, apes, orangutans, bulls, cows, bears, horses, sheep, poultry, mice, rat, fishes, dolphins, whales, sharks and the like, or any animal or organism with detectable nucleic acids. The source may also refer to different parts of the organism, such as interior, exterior, living or dead cells, tissues, liquids and the like. Thus, the sample may be a “biological sample”, which may be a source obtained from living or formerly living, for example any material of an animal like human or other mammals, plants, bacteria, fungi, protozoa or viruses. The source may be in any form, including but not limited to, solid materials such as tissues, cells, cell masses, cell extracts, or biopsy samples, or biological fluids such as urines, blood, saliva, amniotic fluids, exudates from infected or inflammatory areas, or mouthwash containing oral cells, hairs, cerebrospinal fluids and joint fluids, as well as organs. For example, the examples of samples (such as biological samples) may include respiratory tissues, exudates (e.g., bronchoalveolar lavage fluids), living tissue specimens, sputums, peripheral blood, plasmas, serums, lymph nodes, gastrointestinal tissues, feces, urines, cerebrospinal fluids, tissue fluids (e.g., pleural effusions), lavage fluids (e.g., alveoli) or other fluids, tissues or materials. For example, the samples can be treated specimens or materials, for example obtained by treating the samples using filtration, centrifugation, precipitation, or adsorption to a medium (e.g., a matrix or carrier). Other treatments for the samples can include treatments that physically or mechanically disrupt tissue, cell aggregates or cell, thereby releasing intracellular components including nucleic acids into a solution that can contain other components, such as enzymes, buffers, salts, detergents and the like. The samples can also include tissues, for example biopsy tissues (e.g., liquid biopsy tissues), formalin-fixed and paraffin-embedded (FFPE) tissues and the like.

The samples can be isolated from another sample at a different time point, where each sample can be from the same or different sources. The nucleic acid can be from a nucleic acid library, for example a DNA library. The nucleic acid can be a product of nucleic acid purification or separation and/or amplification of nucleic acid molecules in the sample. The nucleic acid for sequencing analysis provided in the present application can contain nucleic acid from one or from two or more samples (for example, 1, 2, 3, 4, 5, 6, 7, 8, 9, 10, 11, 12, 13, 14, 15, 16, 17, 18, 19, 20, 25, 50, 75, 100, 200, 300, 400, 500, 600, 700, 800, 900 or 1,000 or more samples).

The nucleic acids can be processed in a variety of ways during, before, or after the method provided in the present application. For example, the length or mass of nucleic acids can be reduced (e.g., by cleavage, nuclease or restriction enzyme digestion, dephosphorylation, and demethylation), the size or mass of nucleic acids can be increased (e.g., by phosphorylation, reaction with a methylation-specific reagent, ligation with a detectable marker and the like), treatment with a nucleic acid cleavage inhibitor is performed, and the like.

In certain embodiments, untreated nucleic acids are provided for analysis according to the method described in the present application. In certain embodiments, the treated nucleic acids are provided for implementing the method described in the present application. For example, the nucleic acids can be extracted, isolated, purified or amplified from the sample. For example, the nucleic acids can be taken out from the original environment (for example, a natural environment naturally producing the nucleic acid or a host cell exogenously expressing the nucleic acid), so that the nucleic acid is changed from the original environment by “manual” operation. Compared with the content of components in the sample from which the nucleic acid is derived, the isolated nucleic acids generally have fewer non-nucleic acid components (for example, protein, lipid and the like). A composition comprising the isolated nucleic acids can be substantially isolated (for example, about 90%, 91%, 92%, 93%, 94%, 95%, 96%, 97%, 98%, 99% or more than 99% do not contain non-nucleic acid components). In the present application, a purified nucleic acid means that the provided nucleic acid contains fewer non-nucleic acid components compared with the source of the sample from which the nucleic acid is derived. The composition comprising the nucleic acid can be substantially purified (for example, about 90%, 91%, 92%, 93%, 94%, 95%, 96%, 97%, 98%, 99% or more than 99% do not contain other nucleic acids).

### Linear Amplification

The method of the present application may include: treating the sample under conditions that linear amplification can be performed on the nucleic acids derived from the sample, so as to produce the linear amplification products of the nucleic acids.

The nucleic acids in the sample may be isolated before performing the linear amplification, for example, a capture agent and/or a carrier including the capture agent (e.g., a solid carrier, such as a magnetic bead) may be used. For example, the nucleic acids in the sample may be allowed to interact with an immobilized capture probe to capture the nucleic acids onto a solid carrier. Alternatively, nucleic acids in the sample may be allowed to be captured onto a solid carrier as a member of a trimolecular complex, wherein the nucleic acids in the sample and the immobilized capture probe are bridged by target capture oligonucleotide. The solid carrier typically includes a plurality of magnetic or magnetizable particles or beads, which may be manipulated using a magnetic field. The step of isolating the nucleic acids in the sample may also include washing the target capture oligonucleotide: target nucleic acid hybrid to remove undesirable components that may interfere with subsequent amplification.

In some cases, the nucleic acids in the sample are not otherwise isolated prior the linear amplification process.

In the present application, the process for obtaining the linear amplification product may include: contacting the sample with the oligonucleotide capable of binding to the nucleic acids to form an oligonucleotide hybrid; and contacting the oligonucleotide hybrid with an amplification composition under conditions that the linear amplification is allowed to occur, so as to produce the linear amplification product of the nucleic acid.

In certain embodiments, the linear amplification is not a sequence-specific amplification that is only capable of amplifying a particular target sequence in a sample (e.g., the linear amplification is an undifferentiated sequence amplification, also known as a non-preferential or unbiased sequence amplification). For example, the linear amplification may amplify nucleic acids present in a sample (e.g., amplify nucleic acids present in a sample at substantially the same multiple and/or ratio).

In the present application, the linear amplification is performed under conditions that generally do not support exponential amplification of nucleic acids in the sample.

For example, the treatment for obtaining the linear amplification product may include: ligating the nucleic acids derived from the sample with the adaptor sequence to form the adaptor-containing nucleic acids; contacting the adaptor-containing nucleic acids with the oligonucleotide capable of specifically binding to the adaptor sequence to form an oligonucleotide hybrid; and contacting the oligonucleotide hybrid with the amplification composition under conditions that the linear amplification can be performed, so as to produce the linear amplification product of the nucleic acid.

For example, the adaptor sequence may be linked substantially indistinguishably to substantially all nucleic acid molecules in the sample (e.g., it may be linked to one end or both ends of the nucleic acid molecules in the sample (e.g., a DNA molecule, such as cfDNA or ctDNA), e.g., it may be linked to upstream or downstream, i.e., 5′ end or 3′ end of the DNA molecules. The ligation may be performed using a ligase (e.g., a T4 DNA ligase).

The nucleic acids in the sample (e.g., isolated nucleic acids or non-isolated nucleic acids, e.g., isolated or non-isolated DNA, such as cfDNA, in the sample) may be subjected to end repair to obtain blunt ends, before performing the ligation. In addition, deoxyadenosine dA may also be added to the 3′ end of the nucleic acids (e.g., DNA, such as cfDNA or ctDNA) in the sample (e.g., after performing the end repair). In some cases, a phosphate group P may also be added to the 5′ end of nucleic acids (e.g., DNA, such as cfDNA or ctDNA) in the sample.

For example, the ends of nucleic acids (e.g., DNA, such as cfDNA or ctDNA) in the sample may be filled in or nicked using a nucleic acid polymerase in the presence of dNTPs, and a polynucleotide kinase can be used to convert 5′ hydroxyl groups into 5′ phosphate groups and 3′ phosphate groups into 3′ hydroxyl groups. Then, dATP is added to the 3′ end of the nucleic acid molecules (e.g., DNA) using a polymerase having no 3′-5′ exo-activity in the presence of excess dATPs.

In some cases, after the end repair is performed, dA of the 3′ and/or P of the 5′ is added, the nucleic acids (e.g., the 3′ end of the nucleic acids) can be linked to the adaptor sequence to form the adaptor-containing nucleic acids.

For example, the adaptor sequence can be linked directly or indirectly (e.g., by other polynucleotide fragments or sequences) to the 3′ end of the nucleic acid. For example, the 5′ end of the adaptor sequence is linked directly or indirectly to the 3′ end of the nucleic acid.

In the present application, the adaptor sequence can be a double-stranded nucleic acid (e.g., DNA) sequence. In some cases, the adaptor sequence can be a single-stranded nucleic acid sequence, e.g., a single-stranded DNA sequence. The adaptor sequence can comprise about 1-100 nucleotide or base pairs, e.g., about 1-90, 1-80, 1-70, 1-60, 1-50, 1-40, 1-35, 1-30, 1-25, 1-20, 1-15, or 1-10 nucleotide or base pairs.

The adaptor sequence can comprise a promoter sequence of an RNA polymerase, e.g., an SP6 promoter sequence, a T7 promoter sequence, and/or a T3 promoter sequence.

In some cases, the adaptor sequence (e.g., the promoter sequence of the RNA polymerase therein) can comprise one or more modifications, through which the nucleic acid sequence of the adaptor sequence remains unchanged after bisulfite treatment. For example, the promoter sequence of the RNA polymerase (e.g., the SP6 promoter sequence, the T7 promoter sequence, and/or the T3 promoter sequence) can be methylated, e.g., one or more of the bases therein comprise a methylation modification. In some cases, the cytosine contained in the adaptor sequence (e.g., the promoter sequence of the RNA polymerase therein) can be methylation-modified cytosine.

For example, when it is desired to test or identify the methylation status of the nucleic acid in the sample, the adaptor sequence used may comprise a methylated RNA polymerase promoter sequence (e.g., a methylated SP6 promoter sequence, a methylated T7 promoter sequence, and/or a methylated T3 promoter sequence). For example, one or more bases in the RNA polymerase promoter sequence (e.g., the SP6 promoter sequence, the T7 promoter sequence, and/or the T3 promoter sequence) may comprise methylation modification. In some cases, the cytosine contained in the adaptor sequence (e.g., the promoter sequence of the RNA polymerase therein) can be methylation-modified cytosine.

In certain embodiments, the adaptor sequence may comprise a nucleic acid sequence shown in any of SEQ ID NOS: 1-2 and 19-20.

In the method of the present application, the nucleic acid containing the adaptor can be contacted with the oligonucleotide (which is also referred to as a “linear amplification primer” in the present application) capable of specifically binding to the adaptor sequence (e.g., capable of specifically binding to a sense strand of the adaptor sequence or specifically binding to an antisense strand of the adaptor sequence in a case that the adaptor sequence is a double-stranded nucleic acid) under a condition that nucleic acid hybridization is allowed, so as to form an oligonucleotide hybrid. The oligonucleotides capable of specifically binding to the adaptor sequence (“linear amplification primer”) can comprise or not comprise the modified nucleotides. In some cases, the oligonucleotide is not subjected to the methylation modification treatment.

In the present application, the oligonucleotide capable of specifically binding to the adaptor sequence may be at least partially complementary to the adaptor sequence. For example, in a case that the adaptor sequence is double-stranded nucleic acid, the oligonucleotide may be at least partially complementary to the sense strand of the adaptor sequence. In some cases, the oligonucleotide may be at least partially complementary to the antisense strand of the adaptor sequence. For example, the oligonucleotide capable of specifically binding to the adaptor sequence may be substantially complementary (e.g., fully complementary, or at least 80% complementary, e.g., at least 85% complementary, at least 90% complementary, at least 91% complementary, at least 92% complementary, at least 93% complementary, at least 94% complementary, at least 95% complementary, at least 96% complementary, at least 97% complementary, at least 98% complementary, or at least 99% or more complementary, e.g., 100% complementary) to the adaptor sequence. For example, the oligonucleotide may be a single-stranded nucleic acid sequence, for example a single-stranded DNA sequence (e.g., it may comprise about 1-100 nucleotides, for example about 1-90, 1-80, 1-70, 1-60, 1-50, 1-40, 1-35, 1-30, 1-25, 1-20, or 1-15 nucleotides, for example at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, or more).

In certain embodiments, the oligonucleotide capable of specifically binding to the adaptor sequence may comprise a nucleic acid sequence shown as SEQ ID NO: 3.

In some cases, the oligonucleotide hybrid may comprise one or more nucleotides that do not hybridize to the template (e.g., the adaptor sequence). For example, the oligonucleotide hybrid may include one or more mismatched nucleotides (e.g., non-complementary nucleotides) and sometimes non-hybridized 5′ and/or 3′ regions of the nucleic acids. In certain embodiments, the oligonucleotide hybrid may comprise a tag (e.g., a mass distinguishable tag, a sequence tag, a luminescent tag, or a radioactive tag). In certain embodiments, the oligonucleotide hybrid may comprise a capture agent (e.g., a biotin or other capture agents). In certain embodiments, the oligonucleotide hybrid may comprise the terminator nucleotide.

Then, the linear amplification can be performed on the molecules of interest (e.g., a nucleic acid molecule) comprising the oligonucleotide hybrid. In the present application, the linear amplification reaction is performed under conditions that do not support exponential amplification of the nucleic acid sequence of interest (e.g., the nucleic acid molecules in the sample). For example, the linear amplification reaction will generally produce about 2-fold to about 10,000-fold amplification of the nucleic acid sequence, for example, 10-fold to about 10,000-fold amplification of the nucleic acid sequence (e.g., about 50-fold, about 100-fold, about 200-fold, about 300-fold, about 400-fold, about 500-fold, about 600-fold, about 700-fold, about 800-fold, about 900-fold, about 1000-fold, about 1500-fold, about 2000-fold, about 3000-fold, about 5000-fold, about 8000-fold, about 10000-fold or more amplification of the nucleic acid sequence).

In some cases, the linear amplification can be substantially isothermal. In some cases, the linear amplification may include a nucleic acid transcription reaction. For example, the linear amplification may include the nucleic acid transcription reaction, Strand Displacement Amplification reaction (SDA), and/or Rolling Circle Amplification reaction (RCA). For example, the linear amplification may include Non-enzymatic Isothermal Strand Displacement and Amplification (NISDA). In some cases, the linear amplification is non-target specific amplification not for the particular target. For example, the linear amplification can cause nucleic acid molecules in the sample to be amplified substantially indiscriminately, in substantially the same or similar proportions. After the linear amplification, the obtained linear amplification product can comprise RNA encoded by the nucleic acids (e.g., nucleic acid in the sample).

For example, the linear amplification reaction may not involve a thermal cycling process that is characteristic of PCR and other common amplification techniques. For example, the linear amplification reaction may involve contacting the nucleic acid sequence to be amplified (e.g., the molecules of interest comprising the oligonucleotide hybrid) with the amplification composition (e.g., the linear amplification composition), which supports linear amplification of the nucleic acid sequence of interest but lacks at least one component required for exponential amplification of the nucleic acid sequence of interest.

For example, the amplification composition (e.g., the linear amplification composition) may comprise an amplification enzyme that may be selected from the group consisting of a reverse transcriptase, a polymerase, and combinations thereof. The polymerase may be selected from the group consisting of an RNA-dependent DNA polymerase, a DNA-dependent DNA polymerase, a DNA-dependent RNA polymerase, and combinations thereof. In some cases, the amplification composition (e.g., the linear amplification composition) comprises the RNA polymerase. For example, the RNA polymer may be selected from the group consisting of a T7 RNA polymerase, an SP6 RNA polymerase, and a T3 RNA polymerase.

For example, when the adaptor sequence and/or the oligonucleotide hybrid comprises the SP6 promoter sequence, the amplification composition (e.g., the linear amplification composition) may comprise the SP6 RNA polymerase. When the adaptor sequence and/or the oligonucleotide hybrid comprises the T7 promoter sequence, the amplification composition (e.g., the linear amplification composition) may comprise the T7 RNA polymerase. When the adaptor sequence and/or the oligonucleotide hybrid comprises the T3 promoter sequence, the amplification composition (e.g., the linear amplification composition) may comprise the T3 RNA polymerase.

In the present application, the reaction of linear amplification preferably cannot support exponential amplification reaction. For example, it's because that it lacks one or more components required for exponential amplification, and/or there are one or more reagents inhibiting exponential amplification, and/or the conditions under which the reaction is run (e.g., temperature and the like) are not conducive to exponential amplification, etc., For example, the components required for exponential amplification and/or the inhibitor and/or the conditions under which the reaction is run may be selected from the group consisting of amplification oligonucleotides (e.g., amplification oligonucleotides containing the 5′ promoter sequence of DNA polymerase, amplification oligonucleotides of non-promoters, or combinations thereof), enzymes (e.g., polymerase, like DNA polymerase), nucleases (e.g., exonuclease, endonuclease, lyase, ribonuclease, phosphorylase, and glycosylase), enzyme cofactors, chelators (e.g., EDTA or EGTA), ribonucleoside triphosphates (rNTPs), deoxyribonucleoside triphosphates (dNTPs), Mg²⁺, salts, buffers, enzyme inhibitors, blocking oligonucleotides, pH, temperatures, salt concentrations, and combinations thereof.

In the present application, the product obtained after linear amplification reaction is the linear amplification product. For example, the linear amplification product can comprise the RNA molecules obtained by transcribing nucleic acid molecules in the sample.

### Exponential Amplification

In the method of the present application, the linear amplification product can be subjected to exponential amplification to generate the exponential amplification product of the nucleic acid.

In the exponential amplification, the amplification composition (e.g., the exponential amplification composition) can be used, the components of which may include, but are not limited to: nucleotides (e.g., triphosphates nucleotides), modified nucleotides, oligonucleotides (e.g., primer oligonucleotides for polymerase-based amplification and oligonucleotide building blocks for ligase-based amplification), one or more salts (e.g., magnesium-containing salts), one or more buffers, one or more polymerizers (e.g., ligases, and polymerases), one or more nickases (e.g., an enzyme that cleaves one strand of a double-stranded nucleic acid), and one or more nucleases (e.g., exonucleases, endonucleases, and RNA enzyme). Any polymerase suitable for amplification can be used, for example, a polymerase, a DNA polymerase, and an RNA polymerase, and mutant forms of these enzymes, with or without exonuclease activity. Any ligase suitable for binding the 5′ end of one oligonucleotide to the 3′ end of another oligonucleotide can be used. Amplification conditions can also include certain reaction conditions, such as isothermal or temperature cycling conditions. Methods for cycling temperature in the amplification methods are known, such as by thermal cycling device. The term “cycle” generally refers to amplification (e.g., amplification reactions or extension reactions) using a single primer or multiple primers, where temperature cycling is used. In some embodiments, the amplification conditions may also include an emulsifier (e.g., oil) for forming a plurality of reaction compartments in which a single nucleic acid molecular species can be amplified.

For example, the exponential amplification reaction is a reaction performed under conditions that allow for exponential amplification of nucleic acid molecules in the sample or system. For example, the linear amplification product may be contacted with the amplification composition (e.g., the exponential amplification composition) to enable exponential amplification of the nucleic acid under suitable conditions. The exponential amplification composition typically comprises a minimal amount of one or more components required for exponential amplification that are absent from the linear amplification composition. For example, the exponential amplification composition may comprise one or more of the following components or conditions: amplification oligonucleotides, reverse transcriptases, polymerases, nucleases, phosphatases, enzyme cofactors, chelators, ribonucleoside triphosphates (rNTPs), deoxyribonucleoside triphosphates (dNTPs), Mg²⁺, optimal pH, optimal temperatures, salts, and combinations thereof. The polymerase is typically selected from the group consisting of an RNA-dependent a DNA polymerase, a DNA-dependent DNA polymerase, a DNA-dependent RNA polymerase, and combinations thereof. In certain embodiments, the exponential amplification composition comprises the reverse transcriptase and DNA polymerase.

In some cases, the exponential amplification includes Polymerase Chain Reaction (PCR).

In some cases, the exponential amplification reaction is RT-PCR reaction, for example, a one-step RT-PCR reaction, or a two-step RT-PCR reaction. For example, the reverse transcription may provide cDNA templates for PCR amplification and downstream experiments, and the selected reverse transcriptase may have the highest efficiency for all samples, including difficult-to-transcribe RNA samples, such as those that are degraded, have residual inhibitors, or have a high degree of secondary structure. The one-step RT-PCR may include combining a first-strand cDNA synthesis (RT) and a subsequent PCR reaction in a single reaction (e.g., in a single reaction tube). The two-step RT-PCR may include two independent reactions, for example, first performing the first-strand cDNA synthesis (RT) and then amplifying the cDNA obtained in the first step by PCR.

In some cases, the exponential amplification is non-target specific amplification not for the specific target. For example, the obtained amplification products or amplification products can have different nucleic acid sequences from each other (for example, they are each amplified based on a different template sequence, respectively). In specific amplification, the products obtained by amplification are always the same as the template sequences. For example, by using universal primers, the exponential amplification may amplify substantially all nucleic acid molecules (e.g., nucleic acid molecules with different sequences) present in the sample or system in similar proportions or folds. In certain embodiments, the exponential amplification substantially does not use specific amplification primers for the specific target or the specific target sequence, for example, the exponential amplification does not only specifically amplify certain specific nucleic acid molecules or gene fragments present in the sample or reaction system, but substantially does not amplify or amplifies some other nucleic acid molecules or gene fragments present in the sample or the reaction system in a significantly lower proportion.

However, the exponential amplification can be sequence-specific, that is, it can be exponential amplification of the sequences that is common to a plurality of molecules or a variety of molecules in a sample.

Therefore, in certain embodiments, the polymerase-based amplification is achieved by the universal primer. In such a method, a hybridization region that hybridizes with one or more universal primers is introduced into the template nucleic acid. For example, such hybridization reagents can be incorporated with (i) a primer that hybridizes with and is extended from the target nucleic acid, (ii) oligonucleotide that binds (e.g., using ligase ligation) to the target nucleic acid or the product in (i), and/or (iii) the primer having a universal sequence made at the 5′ end of the gene-specific sequence. The amplification method using the universal primer can provide the advantage of amplifying a variety of target nucleic acids, for example, using only one or two amplification primers (also referred to in the present application as “non-target-specific amplification not for a specific target”).

In some embodiments, the exponential amplification includes repeating multiple temperature cycles to amplify the amount of the target nucleic acid. In some embodiments, the amplification reaction is cycled 2 or more times. In some embodiments, the amplification reaction is cycled 10 or more times. In some embodiments, the amplification reaction is cycled about 10, 15, 20, 50, 100, 200, 300 or more times. In some embodiments, the amplification reaction is cycled 20 to 50 times. In some embodiments, the amplification reaction is cycled 30 to 45 times.

In the method of the present application, the strands of a single-stranded nucleic acid target can be amplified (e.g., the exponential amplification can be performed with the RNA molecules as the template), and one or two strands of the double-stranded nucleic acid target can be amplified. In some embodiments, the amplification product (amplified product) is about 10 to about 10,000 nucleotides in length, e.g., about 10 to about 1,000 nucleotides in length, about to about 500 nucleotides in length, about 10 to about 100 nucleotides in length, and sometimes about 10, 11, 12, 13, 14, 15, 16, 17, 18, 19, 20, 25, 30, 35, 40, 45, 50, 55, 60, 65, 70, 75, 80, 80, 85, 90, 95, 100, 125, 150, 175, 200, 225, 250, 275, 300, 325, 350, 375, 400, 425, 450, 475, 500, 600, 700, 800, 900 or 1,000 nucleotides.

### Methylation Test

In certain embodiments, the method of the present application can be used for identifying the presence or absence of one or more methylated target nucleic acids in a sample, or for identifying the content thereof. In some cases, the method of the present application can also be used for identifying the methylation status of nucleotide loci in one or more target nucleic acids in the sample.

For example, the process of performing linear amplification on the nucleic acids in the sample further includes the following steps: treating the sample with the reagent that can modify the unmethylated specific nucleotides to produce other nucleotides. For example, the reagent capable of modifying the unmethylated specific nucleotide to produce other nucleotides can include a reagent that can modify the unmethylated cytosine to produce the uracil. For example, the reagents capable of modifying the unmethylated specific nucleotide to produce other nucleotides can include bisulfite, β-glucosyltransferase, TET enzyme, pyridine borane, and/or A3A deaminase.

For example, the nucleic acid molecules (e.g., DNA) can be denatured and treated with the bisulfite, where C (cytosine) without methylation modification is converted to U (uracil), while 5 mC that is methylated remains 5 mC. Accordingly, after the bisulfite treatment, a specific base position of the target region can be U or C, where U represents that the target region has no methylation modification (and thus converted to U), and C represents that the target region has methylation modification (and thus not converted to U).

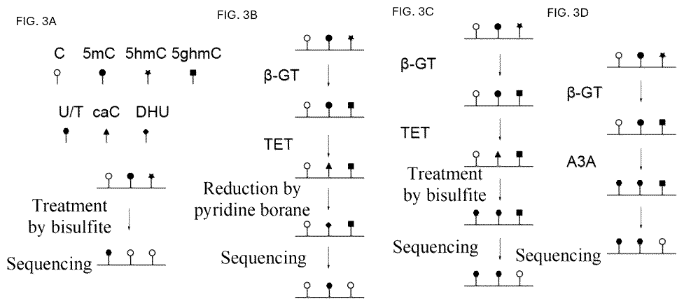

In some cases, the reagents capable of modifying the specific nucleotide that is not methylated to generate other nucleotides may include reagents other than bisulfite (see FIGS. 3A-3D). FIG. 3A shows a process of treatment with bisulfite, during which, C can be deaminated to U, read as T when reading (e.g., sequencing), while 5 mC and 5 hmC are not affected, and still read as C when reading (e.g., sequencing). FIG. 3B shows a process of TAPS sequencing. 5 hmC is protected with sugar using β-glucosyltransferase (β-GT), 5 mC is oxidized to 5 caC using TET enzyme, and 5 caC is reduced to dihydrouracil (DHU) using Pyridine borane, and read as T when reading (e.g., sequencing). FIG. 3C shows a process of a TAB-Seq method. 5 hmC is protected with sugar using β-glucosyltransferase (β-GT), and 5 mC is oxidized to 5 caC using TET enzyme; and in the treatment with bisulfite, C and 5 caC can be deaminated to U, and read as T when reading (e.g., sequencing), while 5 hmC is not affected, and still read as C when reading (e.g., sequencing). FIG. 3D shows a process of an ACE-Seq method. 5 hmC is protected with sugar using β-glucosyltransferase (β-GT), and read as C when reading (e.g., sequencing), and C and 5 mC are deaminated to U using A3A deaminase, and read as T when reading (e.g., sequencing).

In certain embodiments of the method of the present application, step a) (e.g., the step for generating the linear amplification product of the nucleic acid) may include:
ligating the nucleic acids derived from the sample with the adaptor sequence of the present invention to form the adaptor-containing nucleic acids, wherein the adaptor sequence comprises one or more modifications (e.g., methylation modifications, e.g., 5 mC, 5 hmC, 5 ghmC and the like) such that the adaptor sequence has nucleic acid sequence unchanged after treatment with bisulfite; treating the adaptor-containing nucleic acids with the reagent comprising bisulfite to form the converted nucleic acid; contacting the converted nucleic acid with the oligonucleotide capable of specifically binding to the adaptor sequence to form an oligonucleotide hybrid; and contacting the oligonucleotide hybrid with the amplification composition under condition that linear amplification can be performed, so as to generate the linear amplification product of the nucleic acid.

The changes in methylation patterns are often early events in the development and progression of cancer and other diseases. In many cancers, certain genes are improperly inhibited or activated due to abnormal methylation. The ability of methylation patterns to inhibit or activate transcription may be inherited.

Therefore, the method of the present application can be used for testing sequence variations that represent epigenetic changes in a target sequence, for example, changes in the methylation pattern in the target sequence. The covalent addition of methyl to cytosine is mainly present on CpG dinucleotides (microsatellites). Although the function of CpG islands not located in the promoter region still remains to be explored, CpG islands in the promoter region are particularly focused in that their methylation status regulates the transcription and expression of related gene. Methylation in the promoter region results in silencing of gene expression. This silencing is permanent, and persists through a mitotic process. Due to its importance in gene expression, DNA methylation has an effect on the developmental process, imprinting and X-chromosome inactivation, as well as tumorigenesis, aging and inhibition of parasitic DNA. Methylation is believed to be involved in the tumorigenesis of a wide variety of tumors such as lung, breast and colon cancer as well as leukemia. There is also a correlation between methylation and protein dysfunction (Long Q-T syndrome) or metabolic diseases (transient neonatal diabetes mellitus, type-2 diabetes mellitus).

The treatment with bisulfite of DNA (e.g., genomic DNA, cfDNA, or ctDNA) can be used for analyzing the position of the methylated cytosine residue in DNA. The cytosine residue is deaminated to a uracil residue by treating the nucleic acid with bisulfite, while the methylated cytosine remains unchanged. Therefore, by comparing the sequence of the target nucleic acid that is not treated with bisulfite with the sequence of the nucleic acid that is treated with bisulfite according to the method of the present application, the degree of methylation in the methylated nucleic acid and the position where cytosine is methylated can be inferred. Similarly, TAPS sequencing, TAB-Seq, ACE-Seq, and the like can also be used for analyzing the position of the methylated cytosine residue (e.g., 5 mC, 5 hmC, 5 ghmC and the like) in DNA.

### Extension Reaction

In the method of the present application, the exponential amplification product of the nucleic acid is in contact with the extension primer under extension conditions including the terminator nucleotide, thereby generating extended oligonucleotide (i.e., performing extension reaction). The extension reaction of the present application may be a single base extension reaction. For example, the extension reaction is terminated after only the extension primer is subjected to one base extension (e.g., single base extension using the terminator nucleotide).

In the extension reaction of the present application, the nucleic acid polymerase may add one or more nucleotides to the 3′ end of the primer (e.g., oligonucleotide) in a template-specific manner.

Conditions suitable for use in primer extension reactions are known in the art. In general, the primer is annealed (i.e., hybridized) to the target nucleic acid to form a primer-template compound. The primer-template compound is contacted with the DNA polymerase and one or more free nucleotides under suitable conditions, so as to allow addition of the one or more nucleotides to the 3′ end of the primer. In certain embodiments, the primer does not hybridize directly to the nucleic acid sites to be tested (e.g., a target site, such as a methylation modification sites to be identified), but hybridizes to a location adjacent to the location (e.g., the 5′ end of the location). In some embodiments, the primer hybridizes directly to a region adjacent to the target sites (e.g., a methylation modification site to be identified). In some embodiments, the terminator nucleotide may be used in the primer extension reaction to terminate primer extension.

In the present application, the “hybridization site” refers to a specific site on the amplification product or target nucleic acid (e.g., nucleic acid molecules to be detected in the sample). In certain embodiments, the end of the oligonucleotide is adjacent to or substantially adjacent to a site on the amplification product or the target nucleic acid, which site is different in sequence from another amplification product or the target nucleic acid. When there is no nucleotide between the site and the oligonucleotide end, the oligonucleotide end is “adjacent” to the site. In certain embodiments, when there are 1, 2, 3, 4, 5, 6, 7, 8, 9 or 10 nucleotides between the sites and the oligonucleotide ends, the oligonucleotide ends are “substantially adjacent” to the sites.

The extension reaction is typically performed under extension conditions. The extension conditions may/reagents include, but are not limited to: one or more oligonucleotides, extension nucleotides (e.g., nucleotide triphosphates (dNTPs)), terminator nucleotides (e.g., one or more dideoxynucleotide triphosphates (ddNTPs) or acyclic nucleotides), one or more salts (e.g., magnesium-containing salts), one or more buffers (e.g., containing β-NAD, and TritonX-100), and one or more polymerizing agents (e.g., DNA polymerases, and RNA polymerases).

Any suitable extension reaction can be selected and used. For example, the extension reaction can be used for distinguishing SNP alleles or specific methylation sites by incorporating deoxynucleotide and/or terminator nucleotide (e.g., dideoxynucleotide, and acyclic nucleotide) into extension oligonucleotide that hybridizes to a region adjacent to a specific locus in the target nucleic acid. The primers are typically extended by the polymerase. In some embodiments, the oligonucleotide is extended by only one deoxynucleotide or terminator nucleotide (e.g., dideoxynucleotide or acyclic nucleotide) that is complementary to the site to be tested. In some embodiments, the oligonucleotide is extended by incorporating dNTPs and terminated by ddNTPs or acyclic nucleotides, or in certain embodiments, the oligonucleotide is terminated by incorporating ddNTPs or acyclic nucleotides without dNTP extension.

In some embodiments, the oligonucleotide can be extended by any of 5 terminator nucleotides (e.g., ddATP, ddUTP, ddTTP, ddGTP, and ddCTP). The target nucleic acid or corresponding amplification product can be used as the template and can be determined in part which terminator nucleotide in the extension reaction is added to the oligonucleotide. In certain embodiments, other terminator nucleotides (e.g., acyclic nucleotides or terminators) can be utilized.

In certain embodiments, the extension can be performed under isothermal conditions or in a non-isothermal environment (e.g., thermal cycling conditions). One or more target nucleic acids may be extended in the extension reaction, and one or more variants of each target nucleic acid may be extended. The nucleic acids may extend by one or more nucleotides; and in some embodiments, the extension product may have the length of about 10 nucleotides to about 10,000 nucleotides, the length of about 10 to about 1,000 nucleotides, the length of about 10 to about 500 nucleotides, the length of about 10 to about 100 nucleotides, and sometimes the length of about 10, 11, 12, 13, 14, 15, 16, 17, 18, 19, 20, 25, 30, 35, 40, 45, 50, 55, 60, 65, 70, 75, 80, 80, 85, 90, 95, 100, 125, 150, 175, 200, 225, 250, 275, 300, 325, 350, 375, 400, 425, 450, 475, 500, 600, 700, 800, 900, or 1,000 nucleotides. The length of oligonucleotide extension may be determined by incorporating the terminator nucleotides (e.g., ddNTPs), hybridization sites, or other factors. In certain embodiments, the amplification and extension reactions are performed during the same test process.

In some embodiments, the extension reaction involves repeating multiple temperature cycles to amplify the amount of extension product in the reaction. In some embodiments, the reaction cycle is extended 2 or more times. In some embodiments, the reaction cycle is extended 10 or more times. In some embodiments, the reaction cycle is extended about 10, 15, 20, 50, 100, 200, 300, 400, 500, or 600 or more times. In some embodiments, the reaction cycle is extended 20 to 50 times. In some embodiments, the reaction cycle is extended 20 to 100 times. In some embodiments, the reaction cycle is extended 20 to 300 times. In some embodiments, the reaction cycle is extended 200 to 300 times. In some embodiments, the extended reaction cycle is at least 50, 55, 60, 65, 70, 75, 80, 85, 90, 95, or 100 cycles.

In some embodiments, the oligonucleotide (e.g., the extension primer of the present application) that hybridizes to the target nucleic acid (e.g., the exponential amplification product of the present application) is extended by one nucleotide in the presence of the extension composition. The extension composition may comprise one or more of buffers, salts, enzymes (e.g., a polymerases, Klenow and the like), water, templates (e.g., DNA, RNA, amplification product and the like), primers (e.g., oligonucleotides), triphosphate nucleotides, glycerols, macromolecular exclusion molecules, and any other additive used in the art. The extension composition may comprise terminator nucleotides (e.g., dideoxynucleotides (like ddNTPs) or pentacyclic nucleotides), non-terminator or extension nucleotides (e.g., dNTPs), or a mixture of terminator nucleotides and non-terminator nucleotides. The extension composition consists essentially of one or more specific terminator nucleotides, and may contain any other components of the extension composition (e.g., buffers, salts, templates and the like), but does not substantially contain any other terminator nucleotides or triphosphate nucleotides (e.g., dNTPs) in addition to those specified. For example, the extension composition consisting essentially of ddTTPs and ddCTPs does not contain ddATPs, ddGTPs, or any other dNTPs. In some embodiments, the nucleotides in the extension composition are only terminator nucleotides and oligonucleotides that hybridize to the target nucleic acid, and the target nucleic acid or its amplification product is extended by one nucleotide. In some embodiments, the extension composition consists essentially of the terminator nucleotide (e.g., ddNTP, and acyclic nucleotide).

In some embodiments, the terminator nucleotide present in the extension composition (or not present in some embodiments) determines which terminator nucleotides are added to the oligonucleotides. In some embodiments, the extension composition comprises one or more terminator nucleotides (e.g., ddNTPs or acyclic nucleotides). In some embodiments having more than one terminator nucleotides, 2, 3, or 4 strand terminator nucleotides are different terminator nucleotides (i.e., not the same species, e.g., ddA, ddT, ddC, ddG, ddU, acyATP, acyCTP, acyGTP, acyTTP, and acy-bromo-UTP). In some embodiments, the extension composition comprises one or more terminator nucleotides and one or more non-terminator nucleotides (e.g., dNTPs). In some embodiments, the extension composition comprises a terminator nucleotide corresponding to the particular variant (e.g., the first variant, low-quantity variant, or low-abundance variant) and thus is only capable of extending the particular variant. In some embodiments, the extension composition comprises the terminator nucleotide capable of allowing the second variant (e.g., the wild type, high-quantity variant, or high-abundance variant) to be extended, thereby allowing the second variant to be extended. In some embodiments, the method of the present application includes: contacting a hybridized oligonucleotide substance with the extension composition comprising one or more terminator nucleotides under extension conditions, thereby producing an extended oligonucleotide substance, where the oligonucleotide hybridized to the first variant (e.g., the low-abundance variant, less-abundance variant, and low-quantity variant) is extended by the terminator nucleotide, and the oligonucleotide hybridized to the second variant (e.g., wild type, high-abundance variant, and high-quantity variant) is extended by the terminator nucleotide.

For example, the extension primer can be bound specifically to the target nucleic acid (e.g., a specific site or target nucleic acid to be identified). In the present application, the extension primer may comprise at least about 1 nucleotide, e.g., nucleic acid molecules (e.g., single-stranded nucleic acid molecules, for example single-stranded DNA molecules) of at least about 2, 3, 4, 5, 6, 7, 8, 9, 10, 11, 12, 13, 14, 15, 16, 17, 18, 19, 20, 21, 22, 23, 24, or more nucleotides. The extension primer may be substantially fully complementary to its target sequence (e.g., a specific site or target nucleic acid to be identified) (e.g., at least 80% complementary, e.g., at least 85% complementary, at least 90% complementary, at least 91% complementary, at least 92% complementary, at least 93% complementary, at least 94% complementary, at least 95% complementary, at least 96% complementary, at least 97% complementary, at least 98% complementary, or at least 99% or more complementary, e.g., 100% complementary).

In certain embodiments, DNA polymerases for example Therminator™ DNA polymerase from company NEB which is a mutant of a 9°N™ DNA polymerase may be used in the extension reaction (e.g., single-base extension reaction), and the modified enzyme can recognize and incorporate some modified bases such as ddNTPs. Due to the thermostable nature of the enzyme, primer use efficiency can be fully improved through repeated denaturation, annealing, and extension, resulting in increased yields.

Other T7 DNA polymerases, for example genetically engineered T7 DNA polymerases, may also be used in single base extension reaction. For example, the DNA polymerase may be T7 DNA polymerase in a genetically engineered form (e.g., Sequenase). Unlike wild-type enzymes, it may be substantially free of 3′→5′ exonuclease activity. The DNA polymerase may comprise 2 subunits, one of which may be an Escherichia coli thioredoxin protein and the other of which may be a genetically engineered version of the bacteriophage T7 gene 5 protein. The genetic changes in the subunits (e.g., deletion of 28 amino acids by in vitro mutations) may clear all measurable exonuclease activity, but does not change the activity of the DNA polymerase.

For example, the terminator nucleotides may be modified at the 3′ carbon of the pentose moiety. For example, at least one of the terminator nucleotides may be a dideoxynucleotide. For example, the dideoxynucleotide may be selected from the group consisting of ddATP, ddGTP, ddCTP, ddTTP, and ddUTP. In certain embodiments, the terminator nucleotide is an acyclic nucleotide. For example, the acyclic nucleotide may be selected from the group consisting of acy ATP, acyCTP, acyGTP, acyTTP, and acy-bromo-UTP.

For example, the terminator nucleotides include: 1) terminator nucleotide having specificity to other nucleotides generated by modification of specific nucleotide which is not methylated after the treatment, and 2) terminator nucleotide having specificity to the specific nucleotide which is methylated.

### Test

In the present application, the extended oligonucleotide comprises the detectable marker. For example, the detectable marker may be the mass marker. For example, the mass marker may be the mass distinguishable tag. In some cases, the terminator nucleotide may comprise the mass distinguishable tag.

For example, in the method of the present application, the mass marker may be tested by mass spectrometry. The mass spectrometry may be, for example, Matrix-Assisted Laser Desorption Ionization (MALDI) mass spectrometry.

The detectable markers are generally tags that can be distinguished from each other and used for identifying nucleic acids to which the markers are ligated. A variety of types of markers can be selected and used in the method of the present application. For example, oligonucleotides, amino acids, small organic molecules, light emitting molecules, light absorbing molecules, light scattering molecules, luminescent molecules, isotopes, enzymes, and the like can be used as the detectable markers. In certain embodiments, oligonucleotides, amino acids and/or small molecular organic molecules of various lengths, various charge-to-mass ratios, various electrophoretic mobilities (capillary electrophoretic mobilities), and/or various masses can also be used as the detectable markers. Accordingly, fluorophores, radioisotopes, chromophoric agents, luminescent agents, chemiluminescent reagents, light scattering agents, etc. can be used as the markers. The selection of markers depends on the required sensitivity, the difficulty of coupling with nucleic acids, stability requirements, as well as available devices.

In some embodiments, the detectable markers are ligated the terminator nucleotide. In some embodiments, suitable detectable markers can be selected and/or designed to achieve optimal flight performance in mass spectrometry and allow for markers to be distinguished at higher levels of multiplicity. In some embodiments, the markers are fluorescent markers or dyes that are detected by electrophoresis or by performing PCR.

Any suitable test devices can be used for distinguishing detectable markers in the sample. Test devices suitable for testing quality-distinguishable markers include, but are not limited to, certain mass spectrometry and gel electrophoresis devices. Examples of mass spectrometry forms include, but are not limited to, Matrix-Assisted Laser Desorption/Ionization Time-Of-Flight (MALDI-TOF) Mass Spectrometry (MS), MALDI orthogonal TOF mass spectrometry (OTOF MS; two-dimensional), Laser Desorption Mass Spectrometry (LDMS), Electrospray (ES) mass spectrometry, Ion Cyclotron Resonance (ICR) mass spectrometry and Fourier transform mass spectrometry. The method described in the present application is readily applicable to mass spectrometry forms in which the analyte is volatilized and ionized (“ionized MS”, e.g., MALDI-TOF MS, LDMS, ESMS, linear TOF, and OTOF). The orthogonal ion extraction MALDI-TOF and axial MALDI-TOF can yield relatively high resolutions, and thus, relatively high levels of multiplexing. The test devices suitable for detecting light emitting, light absorbing and/or light scattering markers include, but are not limited to, certain photodetectors and photodetectors (e.g., detecting fluorescent, chemiluminescent, absorbing and/or light scattering markers).

The extended product (e.g., extended oligonucleotide of the present application) obtained by the methods of the present application can be tested by a variety of methods. For example, the extension primer (UEP) and/or terminator nucleotide can be labeled with any type of chemical group or moiety that allows for test and/or quantification of signal, including but not limited to, a mass marker, a radioactive molecule, fluorescent molecules, an antibody, an antibody fragment, a hapten, a carbohydrate, a biotin, a biotin derivative, a phosphorescent moiety, a luminescent moiety, an electrochemiluminescent moiety, a moiety that produces an electrochemical signal upon oxidation or reduction, for example a complex of iron, ruthenium or osmium, a colorimetric moiety, a moiety having detectable electron spin resonance, capacitance, dielectric constant or conductivity, or any combination of labels thereof.

In the present application, the “mass distinguishable tag” refers to a marker characterized by mass as a distinguishing feature. In some embodiments, the detectable marker is composed of nucleotides, and sometimes the marker is about 5 nucleotides to about 50 nucleotides in length. In certain embodiments, the detectable marker is a nucleotide complex, which has a length of about 5 nucleotides to about 35 nucleotides. In some embodiments, the detectable marker is peptide, which sometimes has a length of about 5 amino acids to about 100 amino acids. In certain embodiments, the detectable marker is a concatemer of organic molecular units. In some embodiments, the marker is a trityl molecular concatemer. In certain embodiments, the mass distinguishable tag is a chain-terminator nucleotide.

A variety of mass distinguishable tags, such as complexes, amino acids, and/or concatemers, can be selected and used. Different lengths and/or compositions of nucleotide strings (e.g., nucleic acids; and complexes), amino acid strings (e.g., peptides; polypeptides; and complexes), and/or concatemers can be distinguished by mass, and used as markers. Any number of units can be used in the mass distinguishable tag, and the upper and lower units of such units depend in part on the mass window and the resolution of the system for testing and distinguishing such markers. Thus, the length and composition of the mass distinguishable tag can be selected based in part on the mass window and the resolution of the detector for testing and distinguishing the markers.

In certain embodiments, the detectable marker can be released from the nucleic acid product (e.g., extended oligonucleotide). The linkage between the detectable marker and the nucleic acid can be of any type that can be transcribed and cleaved, cleaved and allowed for the test of one or more markers released. In certain embodiments, the marker can be isolated from other moieties of the molecules to which the marker is linked. For example, a linkage that can be cleaved by nuclease (e.g., ribonuclease, and endonuclease); a linkage that can be cleaved by a chemical method; a linkage that can be cleaved by a physical treatment; and a photo-cleavable linker that can be cleaved by light (e.g., o-nitrobenzyl, 6-nitroveratrooxycarbonyl, and 2-nitrobenzyl group). When the light-emitting test system (e.g., matrix-assisted laser desorption ionization (MALDI) mass spectrometry involving laser emission of light) is used, the photo-cleavable linker is advantageous in that cleavage and test are combined together and performed in one step.

For example, according to the method of the present application, when testing the methylation of nucleic acid molecules using a mass spectrometry (e.g., MALDI mass spectrometry), the unmethylated nucleic acid molecules will have one peak at the dA-corresponding position, the methylated nucleic acid molecules will have one peak at the dG-corresponding position, and excess extended primers will appear as additional peaks. Thus, the value of dG/(dA+dG) can represent the proportion or content of the nucleic acid molecule comprising the methylation modification.

### Multiple Test

The method of the present application allows for high throughput test or quantification of the presence, absence or content of multiple target nucleic acids in a sample. Multiplexing refers to simultaneous test of more than one target nucleic acid (or more than one specific site, for example a methylation site). Conventional methods of performing multiplexing reactions in conjunction with mass spectrometry are known (see, for example, WO1997037041A2). Compared to a case that individual target nucleic acids must be subjected to separate mass spectrometry, multiplexing offers the advantage that multiple target nucleic acids and variants thereof (e.g., variants with different sequence variations) can be identified in a single mass spectrum. In some embodiments, the method of the present application can be used in a high throughput, highly automated process for rapid and accurate analysis of target sequences (e.g., DNA methylation, particularly methylation of small amounts of DNA). In some embodiments, the method of the present application can be highly multiplexed in a single reaction. Multiplexing is applicable when the genotype at the locus is unknown, and in some embodiments, the genotype at the locus is known.

In certain embodiments, the number of multiplexed target nucleic acids includes, but is not limited to, about 2-about 1,000, about 2 to about 500, about 2 to about 100, e.g., about 1-5, 5-9, 9-11, 11-13, 13-15, 15-17, 17-19, 19-21, 21-23, 23-25, 25-27, 27-29, 29-31, 31-33, 33-35, 35-37, 37-39, 39-41, 41-43, 43-45, 45-47, 47-49, 49-51, 51-53, 53-55, 55-57, 57-59, 59-61, 61-63, 63-65, 65-67, 67-69, 69-71, 71-73, 73-75, 75-77, 77-79, 79-81, 81-83, 83-85, 85-87, 87-89, 89-91, 91-93, 93-95, 95-97, 97-101, 101-103, 103-105, 105-107, 107-109, 109-111, 111-113, 113-115, 115-117, 117-119, 121-123, 123-125, 125-127, 127-129, 129-131, 131-133, 133-135, 135-137, 137-139, 139-141, 141-143, 143-145, 145-147, 147-149, 149-150, 150-200, 200-250, 250-300, 300-350, 350-400, 400-450, 450-500 or more.

The design method for obtaining resolved mass spectra using multiple analysis may include primer and oligonucleotide design methods and reaction design methods. In terms of primer and oligonucleotide design in multiple analysis, the same primer design overall guidelines are used as for single reactions, for example avoiding spurious triggering and primer dimers, but multiple reactions involve more primers. In addition, analyte peaks in the mass spectrum of a test may be sufficiently resolved from the product of any test with which the test is multiplexed, including pausing peak and any other byproduct peaks. Moreover, the analyte peaks preferably fall within a user-specified mass window, e.g., in the range of 5,000-8,500 Da. In some embodiments, extended oligonucleotides may be designed relative to specific target sequences. In such embodiments, for example, the length can be generally between the range of user-specifiable (e.g., 17-24 bases or 17-26 bases) and generally does not contain any bases that are not defined in the target sequence. Hybridization intensity may sometimes be measured by calculating the sequence-dependent melting (or hybridization/dissociation) temperature Tm. Methods and software for designing extended primers are known and include, for example, SpectroDESIGNER (Sequenom).

In some embodiments, multiplex test provided by the present application are designed for single base extension. For example, oligonucleotides (e.g., extended oligonucleotides in the present application) may be first hybridized with amplification products derived from target nucleic acids (e.g., amplification products of the present invention, for example, exponential amplification products) at a 5′ position of the single base position, and the single base position differs between variants of the target nucleic acid (e.g., target nucleic acids in the presence and absence of methylation modifications).

Diseases, Disease Outcomes and/or Treatment Regimens

The method and product of the present application may be used for the diagnosis of diseases or disorders (including the prognosis of the diseases or symptoms), for example, by a non-invasive method for the diagnosis of the diseases or disorders.

For example, the diseases or disorders may be diseases or disorders associated with changes (e.g., methylation modification or SNP) in genetic materials (e.g., nucleic acid, like DNA) (e.g., determined according to the methylation condition), e.g., tumors or cancers. The genetic materials may be cfDNA, e.g., ctDNA, derived from the samples (e.g., tissues or bodily fluids) of the subject.

In certain embodiments, the method and product of the present application may be used for improving, modulating, or determining a treatment regimen suitable for a particular subject.

### Kits and Systems

In another aspect, the present application provides a kit and/or a system which can be used for implementing the method described in the present application.

The kit can may include: the linear amplification component described in the present application, the exponential amplification component described in the present application, and the extension component described in the present application. In some cases, the linear amplification component, the exponential amplification component and the extension component are not mixed with one another in the kit.

For example, in the kit, the linear amplification component, the exponential amplification component and the extension component can be respectively and independently arranged in a separate package.

For example, the kit can comprise one or more of the following components: a) a reagent which can modify a specific nucleotide which is not methylated to generate other nucleotides; b) one or more nucleic acid polymerases; c) one or more primers; d) a MALDI matrix compound; and e) a MALDI substrate.

In some cases, in the system described in the present application, an apparatus for using a kit can include a thermal cycler and/or a mass spectrometer.

The kit described in the present application generally comprises one or more containers containing one or more components described in the present application. The kit includes any number of one or more components in individual containers, bags, tubes, vials, multiwell plates and the like, or the components can be combined in the containers in various combination modes. The kit can comprise one or more components as follows: (i) one or more nucleotides (for example, terminator nucleotides and/or non-terminator nucleotides), where one or more nucleotides may comprise test markers; (ii) one or more oligonucleotides, where one or more oligonucleotides may comprise test markers (for example, amplification primers, one or more extension primers, and oligonucleotides comprising the markers); (iii) one or more enzymes (for example, polymerases, endonucleases, restriction enzymes and exonucleases); (v) control components (for example, control DNAs, primers, synthesis templates, target nucleic acids and the like); (vi) one or more buffers; and (vii) printed materials (for example, instructions, markers, and the like).

The kits are sometimes used in conjunction with certain methods and may include instructions for performing one or more methods and/or descriptions of one or more compositions. The kits may be used for performing the methods described in the present application. The instructions and/or descriptions may be in tangible form (e.g., paper, etc.) or in electronic form (e.g., computer readable files on tangible media (e.g., optical discs)), and may be included in a kit insert. The kits may also include written instructions that provide an internet location for such instructions or descriptions.

In certain embodiments, the kit of the present application comprises one or more terminator nucleotides, and the terminator nucleotides inhibit the activity of the enzyme on the oligonucleotide when the terminator nucleotides are positioned at the 3′ end of the oligonucleotide. In certain embodiments, the terminator nucleotides are dideoxynucleotides. In certain embodiments, the dideoxynucleotide is selected from ddATP, ddGTP, ddCTP, ddTTP and ddUTP. In certain embodiments, the terminator nucleotide is an acyclic nucleotide. In certain embodiments, the acyclic nucleotide is selected from acyATP, acyCTP, acyGTP, acyTTP and acy-bromo-UTP. In certain embodiments, the terminator nucleotides comprise the mass distinguishable tags.

In certain embodiments, the kit comprises one or more selected from the group consisting of oligonucleotides, polymerases, reverse transcriptases, one or more buffer solutions and one or more reaction controls.

Without intending to be limited by any theory, the Examples in the following are intended only to illustrate the working methods of the apparatus, method and system of the present application, and are not intended to limit the scope of the invention involved in the present application.

### EXAMPLES

### Example 1 Sample Synthesis

### Reference Sequences for Synthesizing Double Strands Respectively are:

Reference sequence 1 (internal reference 1): the forward nucleic acid sequence is shown in SEQ ID NO: 4, and the reverse nucleic acid sequence complementary to the forward nucleic acid sequence is shown in SEQ ID NO: 5. In all reference sequence 1 samples, the content of the nucleic acid molecule containing 5-methylcytosine (i.e., 5 mC) was 0%.

Reference sequence 2 (internal reference 2): the forward nucleic acid sequence is shown in SEQ ID NO: 7, and the reverse nucleic acid sequence complementary to the forward nucleic acid sequence is shown in SEQ ID NO: 8. In all reference sequence 2 samples, the content of nucleic acid molecule containing 5-methylcytosine (i.e., 5 mC) was 25%.

Reference sequence 3 (internal reference 3): the forward nucleic acid sequence is shown in SEQ ID NO: 10, and the reverse nucleic acid sequence complementary to the forward nucleic acid sequence is shown in SEQ ID NO: 11. In all reference sequence 3 samples, the content of nucleic acid molecule containing 5-methylcytosine (i.e., 5 mC) was 50%.

Reference sequence 4 (internal reference 4): the forward nucleic acid sequence is shown in SEQ ID NO: 13, and the reverse nucleic acid sequence complementary to the forward nucleic acid sequence is shown in SEQ ID NO: 14. In all reference sequence 4 samples, the content of nucleic acid molecule containing 5-methylcytosine (i.e., 5 mC) was 75%.

Reference sequence 5 (internal reference 5): the forward nucleic acid sequence is shown in SEQ ID NO: 16, and the reverse nucleic acid sequence complementary to the forward nucleic acid sequence is shown in SEQ ID NO: 17. In all reference sequence 5 samples, the content of nucleic acid molecule containing 5-methylcytosine (i.e., 5 mC) was 100%.

### Example 2 End Repair and Adaptor Ligation 2.1 End Repair and a Tailing

A KAPA Hyper Prep library was used for constructing a kit, and end repair was performed on the reference sequence 1 to the reference sequence 5 in Example 1, respectively. Briefly, an end repair and A tailing reaction mixture was prepared in a PCR tube as follows:

| Component | Volume | |
|---|---|---|
| Reference double-stranded DNA | 1.32 | μl |
| End repair and A tailing enzyme | 0.06 | μl |
| mixture | | |
| End repair and A tailing buffer | 15 | μl |
| Nuclease-free water | 48.62 | μl |
| Total | 65 | μl |

The reaction mixture was uniformly mixed by pipetting, and rapidly centrifuged.

Incubation was carried out in a thermal cycler, and the following setting program was adopted.

| Hot lid 25° C. | Start |
|---|---|
| 20° C. | 15 min |
| 65° C. | 15 min |
| 4° C. | Hold |

### 2.2 Adaptor Ligation

The adaptor stock solution was appropriately diluted, as shown in the table below:
10 ng starting in DNA set:

| Component | Volume |
|---|---|
| Adaptor (methylated T7 adaptor | 5 μl |
| sequence (forward, SEQ ID NO: 1) and | |
| methylated T7 adaptor sequence | |
| (reverse, SEQ ID NO: 2)) | |
| 20 mM Tris-Hcl | 20 μl |

1 ng starting in DNA set (less than 1 ng of DNA was treated as 1 ng):

| Component | Volume |
|---|---|
| Adaptor (methylated T7 adaptor | 1 μl |
| sequence (forward, SEQ ID NO: 1) and | |
| methylated T7 adaptor sequence | |
| (reverse, SEQ ID NO: 2)) | |
| 20 mM Tris-Hcl | 24 μl |

A ligation buffer is thawn, reversed and uniformly mixed, and then put on ice for later use.

A ligation reaction mixture was prepared in the PCR tube that has been subjected to end repair and A-tailing reaction in Example 2.1 as follows:

| Component | Volume | |
|---|---|---|
| End repair and A tailing product | 65 | μl |
| Ligation buffer | 25 | μl |
| DNA ligase | 5 | μl |
| Adaptor diluent | 1 | μl |
| Tris-HCl (20 mM) | 4 | μl |

A pipette was used for gently pipetting to uniformly mix, and the reaction liquid was collected to the bottom of the tube after brief centrifugation.

Incubation was carried out in a thermal cycler, and the following setting program was adopted.

| Temperature | Time |
|---|---|
| 25° C. | Start |
| 20° C. | 15 min |
| 4° C. | Hold |

The ligation product in Example 2.2 was purified by a DNA Clean & Concentrator-5 Kit from Zymo Research according to the description of the instruction to obtain the adaptor-containing nucleic acids.

### Example 3 Nucleic Acid Conversion

The purified adaptor-containing nucleic acids obtained in Example 2.2 was treated by bisulfite through an EZ DNA Methylation-Gold Kit from Zymo Research. Cytosine (C) in DNA sample molecules was converted into uracil (U), and 5-methylcytosine (5 mC) remained unchanged.

### Example 4 Linear Amplification and Reverse Transcription

### 4.1 Linear Amplification

Linear amplification was performed on converted nucleic acid molecules by T7 RNA polymerase.

A linear amplification reaction mixture was prepared according to the following table:

| Component | Volume | |
|---|---|---|
| 10X reaction buffer | 2.2 | μl |
| ATP solution | 2 | μl |
| GTP solution | 2 | μl |
| UTP solution | 2 | μl |
| CTP solution | 2 | μl |
| T7 RNA polymerase mixture | 2 | μl |
| DNA template (converted nucleic acid | 9.8 | μl |
| molecule in Example 3) | | |
| Total volume | 22 | μl |

All components were gently mixed uniformly by the pipette, centrifuged briefly, and incubated at 37° C. for 2 h.

2 μl (100 μM) of linear amplification primer (SEQ ID NO: 3) was added, and placed on ice at 70° C. for 2 min.

The reaction mixture was divided into two tubes, each tube containing 12 μl.

### 4.2 Reverse Transcription Reaction

Reverse transcription was performed on the linear amplification product obtained in Example 4.1. A HiScript III RT SuperMix Kit was used, and the primer for reverse transcription was a nucleic acid sequence shown in SEQ ID NO: 3.

### 4.2.1 Removal of Genomic DNA

The following mixed solution was prepared in an RNase-free centrifuge tube:

| Component | Volume |
|---|---|
| 4 × gDNA wiper Mix | 4 μl |
| Linear amplification | 12 μl |
| product in Example 4.1 | |

The pipettor was used for pipetting to mix uniformly, and incubation was performed at 42° C. for 2 min.

### 4.2.2 Preparation of Reverse Transcription Reaction System

4 μl of 5× HiScript III qRT SuperMix was directly added into the reaction tube in 4.2.1, and gently pipetted by pipette to mix uniformly.

### 4.2.3 Reverse Transcription Reactions Performed According to Following Conditions

| Temperature | Time | |
|---|---|---|
| 50° C. | 15 | min |
| 85° C. | 5 | sec |

The cDNA product obtained after the reverse transcription reaction was purified by using the DNA Clean & Concentrator-5 Kit of Zymo Research.

### Example 5 Exponential Amplification

A PCR mixture was prepared according to the table below.

| Component | Volume | |
|---|---|---|
| 10 × Taq buffer (Mg²⁺ added) | 5 | μl |
| dNTP Mix (10 mM each) | 1 | μl |
| PCR primer (SEQ ID NO: 3)(10 μM) | 4 | μl |
| cDNA obtained in Example 4.2 | 39.5 | μl |
| Taq DNA polymerase (5 U/μl) | 0.5 | μl |

The procedure for performing the PCR reaction was as follows:

| | | | |
|---|---|---|---|
| 95° C. | 3 | min | |
| 95° C. | 15 | sec | 40 cycles |
| 60° C. | 15 | sec | |
| 72° C. | 15 | sec | |
| 72° C. | 5 | min | |

The products of the PCR reaction were purified using an AxyPrep PCR Cleanup Kit (AP-PCR-250G).

### Example 6 Single Base Extension

The PCR product obtained in Example 5 was added into nuclease-free pure water, treated for 10 min at 98° C., and then quickly placed on ice. 1 μl of extension primers (the extension primer of the reference sequence 1 is shown as SEQ ID NO:6, the extension primer of the reference sequence 2 is shown as SEQ ID NO:9, the extension primer of the reference sequence 3 is shown as SEQ ID NO:12, the extension primer of the reference sequence 4 is shown as SEQ ID NO:15, and the extension primer of the reference sequence 5 is shown as SEQ ID NO: 18) was added into each tube of reaction mixture.

| Component | Volume | |
|---|---|---|
| 10 × Buffer | 2 | μl |
| dd ATP | 1 | μl |
| dd GTP | 1 | μl |
| Spec-X (10 μM) | 1 | μl |
| cDNA (PCR product obtained in | 14 | μl |
| Example 5) | | |
| NEB Terminator (5 U/μl) | 1 | μl |
| Total | 20 | μl |

The process for performing the single base extension reaction was as follows:

| | | | |
|---|---|---|---|
| 95° C. | 30 | sec | |
| 95° C. | 30 | sec | 100 cycles |
| 95° C. to 37° C. | 30 | sec | |
| 37° C. | 30 | sec | |
| 37° C. | 10 | min | |

### Example 7 MALDI-TOF Analysis

First, desalination treatment is performed. Desalination can be performed in 2 ways.

In one way, purification was performed through Oligo Clean & Concentrator (D4061) from Zymo Research according to the specification, and the elution was finally performed with purified water.

In another way, using ZipTipμ-C18 pipettor tips (ZTC18S960), the specific steps are as follows:
1) Activate Millipore C 18 ZIP TIP by 50% acetonitrile neutral solution, and aspirate and drain for 5 to 10 times;
2) Equilibrate C18 ZIP TIP by 0.1 M of tetraethylammonium acetate tetrahydrate TEAA (pH=7.0), and aspirate and drain for 5 to 10 times;
3) Add 1 μl of 2 M TEAA (pH=7.0) into a 20 μl of single base extension system in Example 6, adsorb by C18 ZIP TIP, and aspirate and drain for 5 to 10 times;
4) Wash with 0.1% of 0.1 M TEAA (pH=7.0), and aspirate and drain for 3 times;
5) Wash with ultrapure water, and aspirate and drain for 3 times;
6) Pipette 4 μl of solution containing 50% acetonitrile to elute the nucleic acid fragment for mass spectrometry, and aspirate and drain for 5 to 10 times in an empty sample tube; and
7) Wash the ZIP TIP column by aspirating and draining for 5-10 times with 70% acetonitrile neutral solution.

The mass spectrometry analysis was performed by a Mass ARRAY mini-analyzer (MALD1-TOF mass spectrometer) to obtain a test result.

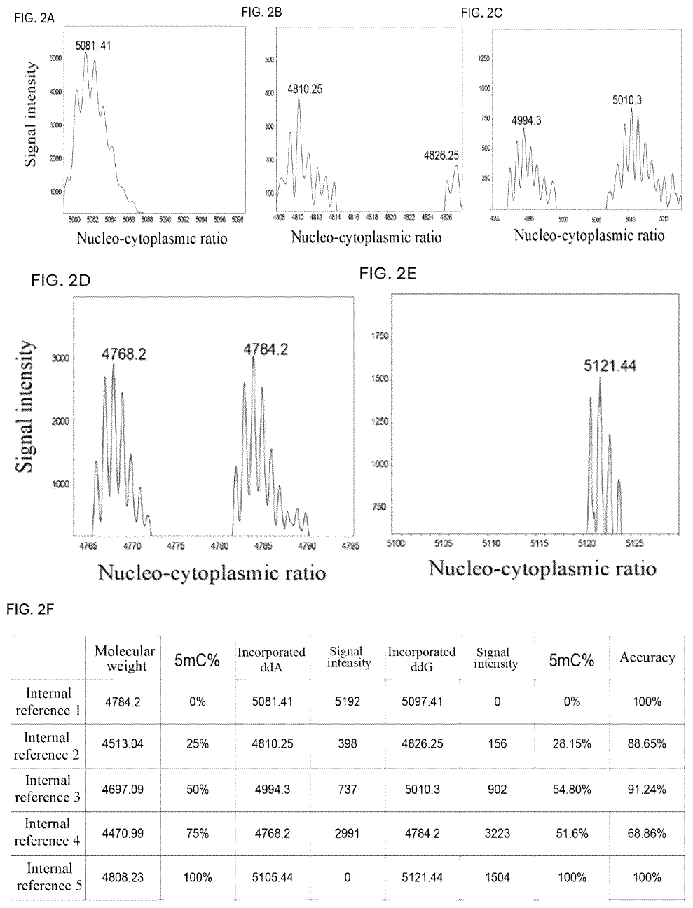

Generally, 1 ng of cfDNA can be extracted from about 2 ml of whole blood. By means of the method of the present application, about 1 ng or less of DNA samples and methylation modification in the samples can be effectively detected. Test results of the internal reference 1 to the internal reference 5 are shown in FIGS. 2A-2E respectively, and the results are summarized in FIG. 2F. FIG. 2F shows the molecular weight of each internal reference specific primer before and after single base extension, and the accuracy of the methylation content of the specific site calculated by the micro nucleic acid mass spectrum. It can be seen that the test accuracy of the internal reference 1 to the internal reference 5 is at least about 65% or higher.

### Example 8 Amplification Reaction Based on Another Strand Displacement Amplification

### Methylated NISDA Linear Amplification Technology

A adaptor could be treated as double-stranded DNA, and the adaptor could contain at least one nickase that can recognize a conserved sequence and a corresponding number of protected bases; or an endonuclease recognition sequence of which the activity was not influenced by methylation modification of cytosine ribonucleoside. Moreover, in the nickase recognition sequence of the adaptor, a C base on at least one strand was protected by 5-position methylation. The 3′ end of one strand of the adaptor could be provided with an additional T tail which was not matched with a complementary strand so as to facilitate end repair, and the 3′ end was ligated with a DNA fragment after A base tailing. If the adaptor contained the endonuclease recognition sequence, the nucleotides on one of chains on both sides or near both sides of a cleavage position corresponding to the endonuclease was subjected to Phosphorothioate modification or other modifications capable of resisting the activity of the endonuclease, so that the effects were achieved that one of the double strands of the DNA was cleaved by the endonuclease, and the other strand kept the original complete state.

The double-stranded adaptor having the characteristics was ligated to a DNA fragment, for example, by using a DNA ligase system.

The ligated DNA fragment was treated by sulfite, so that C base which was not methylated was converted into the T base, and the treated product should be a single-stranded DNA.

A complementary single-stranded primer for the DNA sequence of one strand of the double-stranded adaptor after treatment by the sulfite was added; and after being annealed, it formed a partial double-stranded structure with the single-stranded DNA, and the partial double-stranded DNA at least included a nickase or endonuclease recognition sequence and its corresponding number of protected bases.

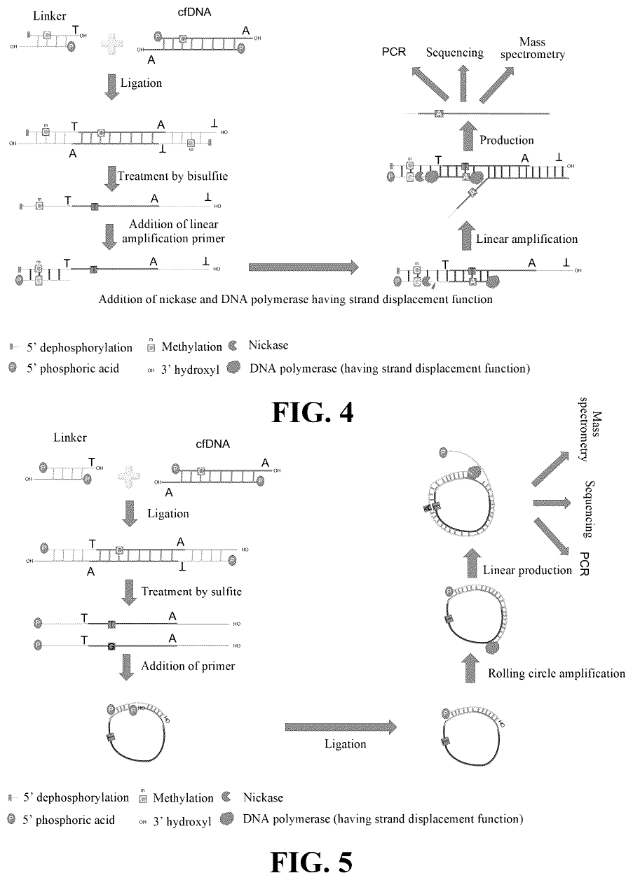

The DNA polymerase containing at least the nickase (or endonuclease) and having a strand displacement function was added, and amplification based on the nicked DNA was initiated; and DNA single-stranded binding protein could be added into the system to stabilize the reaction system. As shown in FIG. 4, the obtained linear amplification product was used for the test method of nucleic acid of the present application, for example downstream PCR reaction, sequencing or nucleic acid mass spectrometry test, and the test accuracy of the methylation content at the specific sites could be realized.

### Example 9 Linear Amplification Reaction Based on Another Rolling Ring Amplification

### Methylated RCA Linear Amplification Technology

A adaptor could be double-stranded DNA, the length of the adaptor could be not less than 5 bp, and the adaptor didn't contain a C base or contained a C base but was protected by methylation at the 5th position. The 3′ end of one strand of the adaptor could be provided with an additional T tail which was not matched with a complementary strand so as to facilitate end repair, and the 3′ end was ligated with a DNA fragment after A base tailing.

The double-stranded adaptor with the characteristics was ligated with the DNA fragment, and a DNA ligase system could be used.

The ligated DNA fragment was treated by sulfite, so that C base which was not methylated was converted into the T base, and the treated product should be a single-stranded DNA.

Single-stranded primers which were complementary with the sequences of both strands of the double-stranded adaptor were added, and annealed, so that both ends of the single-stranded DNA with the adaptor, which was subjected to C-T conversion, were combined with the single-stranded primers.

An enzyme with DNA ligation function was added so that both ends of the single-stranded DNA with the adaptor were ligated to form circular DNA.

Then, DNA polymerase with strand displacement function was added to initiate the rolling circle amplification; and a DNA single-stranded binding protein could be added into the system to stabilize a reaction system.

As shown in FIG. 5, the rolling circle amplification product is a linear amplification product. The obtained linear amplification product is used for the nucleic acid test method of the present application, for example downstream PCR reaction, sequencing or nucleic acid mass spectrometry test, and the test accuracy of the methylation content at a specific site can be realized.

### Example 10 Test Results of Clinical Sample

The methylation status of septin9 in 6 ml of plasma of a patient with colorectal cancer was tested according to the following steps.

The cfDNA in the plasma was extracted by an extraction kit from Epican Technology Limited, and end repair and T7 methylation adaptor ligation were performed on the cfDNA obtained by a KAPA Hyper Prep library construction kit according to the test method described in the present application. Then the cfDNA subjected to adaptor ligation was purified by DNA Clean & Concentrator-5 (Zymo Research). Then treatment by sulfite was performed on the purified product by the EZ DNA Methylation-Gold (Zymo Research) kit. The sulfite treated product was then purified. Linear amplification was performed on the purified product subjected to sulfite treatment using T7 RNA polymerase and reverse transcriptase, and the specific operation was shown in Example 4.

The linear amplification product was subjected to PCR amplification by combinations of 7 pairs of PCR amplification primer which were specially designed for methylation sites of the promoter region of human genome septin9, the product was cleaned and purified, and the PCR amplification procedure and the cleaning and purifying operations were shown in Example 5.

### Primer Sequences

| | | | SEQ |
|---|---|---|---|
| Site | Primer | Sequence | ID NO |
| chr17: 75314424 | Forward | TGAGAGTATTTTGGATGGTTTAGGTGG | 21 |
| | Reverse | CTACAAATCTCTCCAAACCCCATCTCC | 22 |
| | Extension | TCAAATCTCCCTCTACCCCC | 23 |
| chr17: 75253886 | Forward | GGATAGGTATTGGGGTTGAAGTTGT | 24 |
| | Reverse | TCCCCTCAACTTCATTTCCTAAAAC | 25 |
| | Extension | AAAAAACACCACTCCAAACC | 26 |
| chr17: 75458524 | Forward | GAGTTTAGGAGTTTGAGATTAGTTTG | 27 |
| | Reverse | CACTCTTAAATCCCAACTCAAACTC | 28 |
| | Extension | CTACAAACATATACCACCCC | 29 |
| chr17: 75418017 | Forward | GTAGTTTTAGTTATTTGGTAGGTTGAGATG | 30 |
| | Reverse | CAATCTCTCTCTATCACCCAAACTAAAATT | 31 |
| | Extension | ATCCCACTCACTATAATCTC | 32 |
| chr17: 75253759 | Forward | GGTTTTATGAGTATTGTTGGGTTTGGAT | 33 |
| | Reverse | CAACACATAAATTCCTAACCCCCAAAT | 34 |
| | Extension | AAATAAAAACCAATACAACC | 35 |
| chr17: 75391725 | Forward | GGGTAGAAAATAATGATTTTTGGATGGAAT | 36 |
| | Reverse | ATTCCCTAAACATACTAAATAATCCCCAT | 37 |
| | Extension | TATAATAAACAACACCCACC | 38 |
| chr17: 75348262 | Forward | GGTTTTATTATGTTAGTTAGGATGGTTTTG | 39 |
| | Reverse | ATCCAAAATCAACCACATACAATAACTC | 40 |
| | Extension | AACCAAAACAATTAAATCAC | 41 |

The forward and reverse are PCR amplification primers of corresponding sites, and the extension is extension primers of corresponding sites.

For the number of primer pairs used for PCR amplification of Septin9, there were seven sites and seven pairs of primers in total, and the concentration of the primers in each reaction was 2.5 μM. For the number of extension primers used for Septin9, there were seven sites and seven primers in total, and the concentration of the primers in each reaction was 0.5 μM.

After PCR amplification was completed, 7 extension primer combinations which were specially designed for the methylation site of the eptin9 promoter region were added into the reaction system shown above, and the extension reaction system was carried out according to the procedure shown in Example 6.

According to Example 7, the extension reaction product above was desalted and purified.

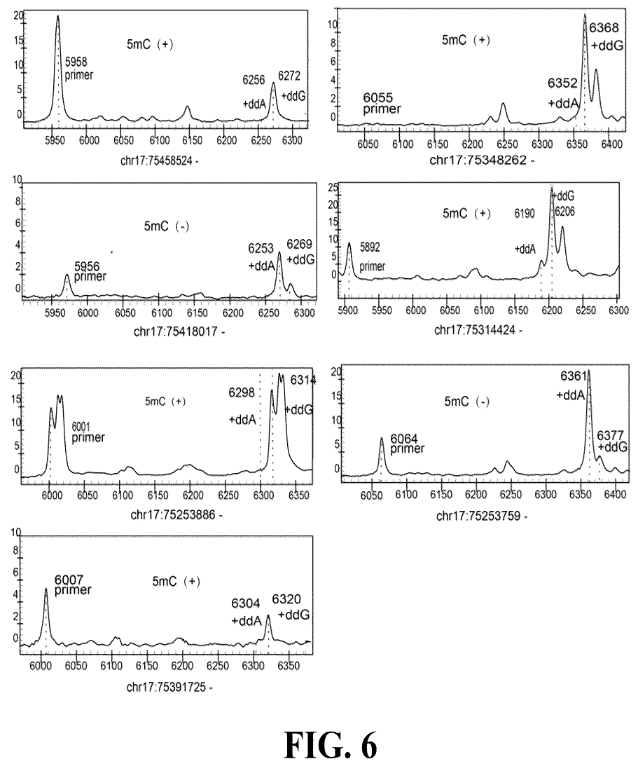

A nucleic acid time-of-flight mass spectrometer was used for analyzing the desalted product, with the result shown in FIG. 6, and methylation modifications of seven methylation site of septin9 in a patient were all detected.

The result shows that the methylation test method provided in the present application can realize relatively high accuracy in clinical detection.

The foregoing detailed description are provided by way of explanation and illustration and are not intended to limit the scope of the attached claims. Various modifications to the embodiments described herein will be apparent to those of ordinary skill in the art and remain within the scope of the appended claims and their equivalents.

1. A method for identifying the presence or absence of one or more target nucleic acids in a sample, or for identifying the content thereof, comprising:
a) treating the sample under a condition that nucleic acids derived from the sample is capable of being subjected to linear amplification, so as to produce a linear amplification product of the nucleic acids;
b) performing exponential amplification on the linear amplification product to produce an exponential amplification product of the nucleic acids;
c) contacting the exponential amplification product of the nucleic acids with an extension primer under extension conditions comprising a terminator nucleotide to produce an extended oligonucleotide; and
d) analyzing the extended oligonucleotide to identify the presence or absence of one or more target nucleic acids in the sample, or to identify the content thereof.

2. The method according to claim 1, wherein step a) comprises:
contacting the sample with an oligonucleotide that is capable of binding to the nucleic acids to form an oligonucleotide hybrid; and
contacting the oligonucleotide hybrid with an amplification composition under a condition that linear amplification is allowed to occur, so as to produce the linear amplification product of the nucleic acids.

3. The method according to claim 1, wherein step a) comprises:
ligating the nucleic acids derived from the sample with a adaptor sequence to form the adaptor-containing nucleic acids;
contacting the adaptor-containing nucleic acids with an oligonucleotide that is capable of specifically binding to the adaptor sequence to form an oligonucleotide hybrid; and
contacting the oligonucleotide hybrid with an amplification composition under a condition that linear amplification is allowed to occur, so as to produce the linear amplification product of the nucleic acids.

4. The method according to claim 3, wherein the adaptor sequence comprises a promoter sequence of an RNA polymerase.

5. The method according to claim 3, wherein the adaptor sequence comprises an SP6 promoter sequence, a T7 promoter sequence and/or a T3 promoter sequence.

6. The method according to claim 3, wherein the oligonucleotide that is capable of specifically binding to the adaptor sequence is at least partially complementary to the adaptor sequence.

7. The method according to claim 4, wherein the oligonucleotide that is capable of specifically binding to the adaptor sequence comprises a sequence complementary to the promoter sequence of the RNA polymerase.

8. (canceled)

9. The method according to claim 3, wherein the adaptor sequence comprises one or more modifications through which the nucleic acid sequence of the adaptor sequence remains unchanged after the adaptor sequence is subjected to bisulfite treatment.

10. The method according to claim 3, wherein the adaptor sequence comprises methylation modification of one or more bases.

11-15. (canceled)

16. The method according to claim 1, wherein the linear amplification comprises a nucleic acid transcription reaction, a strand displacement amplification reaction and/or a rolling circle amplification reaction.

17. The method according to claim 1, wherein the linear amplification is non-target specific amplification not for a specific target.

18. (canceled)

19. The method according to claim 1, wherein the RNA polymerase is used in the linear amplification.

20. The method according to claim 19, wherein the RNA polymerase comprises a T7 RNA polymerase, an SP6 RNA polymerase, and/or a T3 RNA polymerase.

21. The method according to claim 1, wherein step a) further comprises:
treating the sample by a reagent capable of modifying specific nucleotide which is not methylated to generate other nucleotides.

22. (canceled)

23. The method according to claim 21, wherein the reagent capable of modifying the specific nucleotide which is not methylated to generate other nucleotides comprises a bisulfite, a β-glucosyltransferase, a TET enzyme, a pyridine borane and/or a A3A deaminase.

24. (canceled)

25. The method according to claim 1, being used for identifying the presence or absence of one or more methylated target nucleic acids in a sample, or for identifying the content thereof.

26-44. (canceled)

45. The method according to claim 1, wherein the nucleic acids derived from the sample comprises a cfDNA and/or a genomic gDNA.

46.-55. (canceled)

56. A method for identifying a biomarker associated with a disease, a disease outcome, and/or a treatment regimen outcome, comprising the following steps:
i) identifying the presence or absence of one or more target nucleic acids derived from one or more samples, or identifying the content thereof according to the method of claim 1, the one or more samples being derived from one or more subjects having a known disease, a disease outcome, and/or a treatment regimen outcome;
ii) identifying the presence or absence of one or more target nucleic acids derived from one or more samples, or identifying the content thereof according to said method, the one or more samples being derived from a normal subject; and
iii) identifying a difference between the presence or absence of one or more target nucleic acids, or the content thereof in step i) and the presence or absence of the one or more target nucleic acids, or the content thereof in step ii), and accordingly identifying the difference as a biomarker associated with the disease, disease outcome, and/or treatment regimen outcome.

57. A method for identifying methylation associated with a disease, a disease outcome, and/or a treatment regimen outcome, comprising the following steps:
i) identifying methylated or unmethylated nucleotides in one or more target nucleic acids derived from one or more samples according to the method of claim 1, the one or more samples being derived from one or more subjects having a known disease, a disease outcome, and/or a treatment regimen outcome;
ii) identifying methylated or unmethylated nucleotides in one or more target nucleic acids derived from one or more samples according to said method, the one or more samples being derived from a normal subject; and
iii) identifying a difference between the methylated or unmethylated nucleotides in the one or more target nucleic acids in step i) and the methylated or unmethylated nucleotides in the one or more target nucleic acids in step ii), and accordingly identifying the differentiated methylated or unmethylated nucleotides as methylation associated with the disease, disease outcome, and/or treatment regimen outcome.

58. (canceled)

59. A kit for identifying the presence or absence of one or more target nucleic acids in a sample, or for identifying the content thereof, comprising:
a linear amplification component;
an exponential amplification component; and
an extension component comprising a terminator nucleotide, a thermostable elongase, and an extension primer.

60-64. (canceled)


SEQUENCE LISTING (41 sequences)

SEQ ID NO: 1 - DNA, 34 bp, synthetic construct
  modified_base 6: mod_base = m5c
  modified_base 9: mod_base = m5c
  modified_base 11: mod_base = m5c
  modified_base 13: mod_base = m5c
  modified_base 22: mod_base = m5c
  modified_base 23: mod_base = m5c
  modified_base 24: mod_base = m5c
  modified_base 26: mod_base = m5c
  modified_base 32: mod_base = m5c
  source 1..34: mol_type = other DNA
taatacgactcactataggatccctcaataacgt

SEQ ID NO: 2 - DNA, 33 bp, synthetic construct
  modified_base 1: mod_base = m5c
  modified_base 15: mod_base = m5c
  modified_base 16: mod_base = m5c
  modified_base 27: mod_base = m5c
  source 1..33: mol_type = other DNA
cgttattgagggatcctatagtgagtcgtatta

SEQ ID NO: 3 - DNA, 18 bp, synthetic construct
  source 1..18: mol_type = other DNA
taatacgactcactatag

SEQ ID NO: 4 - DNA, 40 bp, synthetic construct
  source 1..40: mol_type = other DNA
gtaagctgactttatggcctcgaaaccaccgagccggaac

SEQ ID NO: 5 - DNA, 40 bp, synthetic construct
  source 1..40: mol_type = other DNA
gttccggctcggtggtttcgaggccataaagtcagcttac

SEQ ID NO: 6 - DNA, 16 bp, synthetic construct
  source 1..16: mol_type = other DNA
ccaactcaataatttc

SEQ ID NO: 7 - DNA, 40 bp, synthetic construct
  modified_base 24: mod_base = m5c
  source 1..40: mol_type = other DNA
ttcatgaaatcccgggaggtaaacgggcatttcagttcaa

SEQ ID NO: 8 - DNA, 40 bp, synthetic construct
  source 1..40: mol_type = other DNA
ttgaactgaaatgcccgtttacctcccgggatttcatgaa

SEQ ID NO: 9 - DNA, 15 bp, synthetic construct
  source 1..15: mol_type = other DNA
taaactaaaataccc

SEQ ID NO: 10 - DNA, 40 bp, synthetic construct
  modified_base 20: mod_base = m5c
  source 1..40: mol_type = other DNA
ccgtcactgcataaaccatcgggagagcaggcggtacgca

SEQ ID NO: 11 - DNA, 40 bp, synthetic construct
  source 1..40: mol_type = other DNA
tgcgtaccgcctgctctcccgatggtttatgcagtgacgg

SEQ ID NO: 12 - DNA, 16 bp, synthetic construct
  source 1..16: mol_type = other DNA
taccacctactctccc

SEQ ID NO: 13 - DNA, 41 bp, synthetic construct
  modified_base 23: mod_base = m5c
  source 1..41: mol_type = other DNA
sactttcgtcgcgatagatgatcggggattcagtaacattc

SEQ ID NO: 14 - DNA, 41 bp, synthetic construct
  source 1..41: mol_type = other DNA
sgaatgttactgaatccccgatcatctatcgcgacgaaagt

SEQ ID NO: 15 - DNA, 15 bp, synthetic construct
  source 1..15: mol_type = other DNA
tattactaaatcccc

SEQ ID NO: 16 - DNA, 40 bp, synthetic construct
  modified_base 19: mod_base = m5c
  source 1..40: mol_type = other DNA
taacttccggagccacaccggtgcaaacctcagcaagcag

SEQ ID NO: 17 - DNA, 40 bp, synthetic construct
  source 1..40: mol_type = other DNA
ctgcttgctgaggtttgcaccggtgtggctccggaagtta

SEQ ID NO: 18 - DNA, 16 bp, synthetic construct
  source 1..16: mol_type = other DNA
tactaaaatttacacc

SEQ ID NO: 19 - DNA, 34 bp, synthetic construct; identical to SEQ ID NO: 1
  source 1..34: mol_type = other DNA

SEQ ID NO: 20 - DNA, 33 bp, synthetic construct; identical to SEQ ID NO: 2
  source 1..33: mol_type = other DNA

SEQ ID NO: 21 - DNA, 27 bp, synthetic construct
  source 1..27: mol_type = other DNA
tgagagtattttggatggtttaggtgg

SEQ ID NO: 22 - DNA, 27 bp, synthetic construct
  source 1..27: mol_type = other DNA
ctacaaatctctccaaaccccatctcc

SEQ ID NO: 23 - DNA, 20 bp, synthetic construct
  source 1..20: mol_type = other DNA
tcaaatctccctctaccccc

SEQ ID NO: 24 - DNA, 25 bp, synthetic construct
  source 1..25: mol_type = other DNA
ggataggtattggggttgaagttgt

SEQ ID NO: 25 - DNA, 25 bp, synthetic construct
  source 1..25: mol_type = other DNA
tcccctcaacttcatttcctaaaac

SEQ ID NO: 26 - DNA, 20 bp, synthetic construct
  source 1..20: mol_type = other DNA
aaaaaacaccactccaaacc

SEQ ID NO: 27 - DNA, 26 bp, synthetic construct
  source 1..26: mol_type = other DNA
gagtttaggagtttgagattagtttg

SEQ ID NO: 28 - DNA, 25 bp, synthetic construct
  source 1..25: mol_type = other DNA
cactcttaaatcccaactcaaactc

SEQ ID NO: 29 - DNA, 20 bp, synthetic construct
  source 1..20: mol_type = other DNA
ctacaaacatataccacccc

SEQ ID NO: 30 - DNA, 30 bp, synthetic construct
  source 1..30: mol_type = other DNA
gtagttttagttatttggtaggttgagatg

SEQ ID NO: 31 - DNA, 30 bp, synthetic construct
  source 1..30: mol_type = other DNA
caatctctctctatcacccaaactaaaatt

SEQ ID NO: 32 - DNA, 20 bp, synthetic construct
  source 1..20: mol_type = other DNA
atcccactcactataatctc

SEQ ID NO: 33 - DNA, 28 bp, synthetic construct
  source 1..28: mol_type = other DNA
ggttttatgagtattgttgggtttggat

SEQ ID NO: 34 - DNA, 27 bp, synthetic construct
  source 1..27: mol_type = other DNA
caacacataaattcctaacccccaaat

SEQ ID NO: 35 - DNA, 20 bp, synthetic construct
  source 1..20: mol_type = other DNA
aaataaaaaccaatacaacc

SEQ ID NO: 36 - DNA, 30 bp, synthetic construct
  source 1..30: mol_type = other DNA
gggtagaaaataatgatttttggatggaat

SEQ ID NO: 37 - DNA, 29 bp, synthetic construct
  source 1..29: mol_type = other DNA
attccctaaacatactaaataatccccat

SEQ ID NO: 38 - DNA, 20 bp, synthetic construct
  source 1..20: mol_type = other DNA
tataataaacaacacccacc

SEQ ID NO: 39 - DNA, 30 bp, synthetic construct
  source 1..30: mol_type = other DNA
ggttttattatgttagttaggatggttttg

SEQ ID NO: 40 - DNA, 28 bp, synthetic construct
  source 1..28: mol_type = other DNA
atccaaaatcaaccacatacaataactc

SEQ ID NO: 41 - DNA, 20 bp, synthetic construct
  source 1..20: mol_type = other DNA
aaccaaaacaattaaatcac